(19) United States
(12) Patent Application Publication
(10) Pub. No.: US 2026/0272781 A1
(43) Pub. Date: Sep. 17, 2026

(54) PLATE-LIKE HOLDING PART

(71) Applicant: Datwyler Pharma Packaging Belgium, Alken (BE)

(72) Inventors: Nicholas Frank BRUNO, Pottstown, PA (US); Alexis Marie Adolphe DECHELETTE, Elkins Park, PA (US); Bart BIESMANS, Kuringen (BE)

(73) Assignee: Datwyler Pharma Packaging Belgium, Alken (BE)

(21) Appl. No.: 19/080,421

(22) Filed: Mar. 14, 2025

(51) Int. Cl. A61J 1/16

(52) U.S. Cl. CPC A61J 1/16

(57) ABSTRACT

A plate-like holding part for holding parts, such as stoppers or caps for pharmaceutical vessels, has a plate part having a plate plane, wherein a receptacle extending perpendicular to the plate plane is formed on a first plate side of the plate part, with a circular ring-shaped receptacle wall. The receptacle wall is formed with a step, with a first wall section penetrated by the plate plane and a second wall section adjoining the step, which begins at a distance from the plate plane. A front surface of the receptacle wall, which runs in an approximately plate-parallel manner, has several protrusions distributed over the circumference. A support wall extends between the receptacles, which has a second height. The second height is larger than the first height, and the support wall has a longitudinal extension, which corresponds to half of a free diameter or more of a receptacle.

### FIELD OF TECHNOLOGY

The invention relates to a plate-like holding part for holding parts, such as stoppers or caps for pharmaceutical vessels, with a plate part having a plate plane, wherein a receptacle extending perpendicular to the plate plane is formed on a first side of the plate part, with a circular ring-shaped receptacle wall.

The invention also relates to a plate-like holding part for holding parts, such as stoppers or caps for pharmaceutical vessels, with a plate part having a plate plane, wherein a plurality of receptacles extending perpendicular to the plate plane is formed on the plate part, each with a circular ring-shaped receptacle wall, wherein the receptacle walls have an essentially identical first height.

### PRIOR ART

Holding parts of the type in question are known, for example, for receiving elastomer stoppers or aluminum crimp caps, which, for closing pharmaceutical vessels, preferably pre-sterilized filling cartridges, are optionally supplied to them automatically. With regard to the holding part, it can be a tray or nest structure.

Reference is made, for example, to the EP 3 450 349 A1 (also published as US 2019/0 125 473 A1).

### SUMMARY OF THE INVENTION

With regard to the above-described prior art, the invention has the object of advantageously designing a holding part of the type in question.

A possible solution of the object is at hand according to a first idea of the invention in the case of a holding part, which focusses on that the receptacle wall is formed with a step, with a first wall section penetrated by the plate plane and a second wall section adjoining the step, which begins at a distance from the plate plane.

Advantageously, when a part, in particular a stopper, is held in the holder, the part may be supported solely or at least predominantly solely in the area of the step. The contact surface of the part to be received and to be held is reduced so that a sufficient sterilization can be carried out in the receiving position. It can in particular be attained thereby that the stopper front surface facing the vessel interior after attaching the stopper to the pharmaceutical vessel, but also, if necessary, the side surface that faces the inside of the neck of the vessel after assembly with the vessel, and also, if necessary, a part of the bottom surface of a flange of the stopper, is completely exposed in the receptacle and is thus subjected completely to the sterilization process. Independently of this or in addition, an advantageous stable design is also achieved.

In one possible embodiment, the distance between the step and the plate plane can correspond to approximately one tenth to approximately one third, furthermore for example approximately one fifth of an inner diameter of the second wall section. This distance between the step and the plate plane can also be very small, for example 0.2 times or less of the diameter, and possibly even close to or equal to zero.

According to a further solution, a front surface of the receptacle wall, which runs in an approximately plate-parallel manner, can have several protrusions distributed over the circumference.

This can also result in an advantageously small contact surface of the part to be received. For example, a receptacle designed in this way can serve the purpose of holding caps of pharmaceutical vessels (vials) in a transport-safe manner. These caps can support themselves point by point with the inner side of the cap top on the protrusions. The protrusions distributed around the circumference result in ceiling areas of the cap to be held that are exposed in the circumferential direction between two protrusions, which are exposed in particular for carrying out a sterilization process. Such caps are known, for example, as aluminum crimp caps, which, for closing pharmaceutical vessels, preferably pre-sterilized filling cartridges or vials, are optionally supplied to them automatically.

The object can also be solved in that a support wall extends between the receptacles, which has a second height, wherein the second height is larger than the first height, and that the support wall has a longitudinal extension, which corresponds to half of a free diameter or more of a receptacle.

The support wall provides for a stacking of several holding parts one on top of the other, wherein the support walls can provide a spacing, which is vertical in the usual use position, of two holding parts arranged one on top of the other. Due to this spacing, it can be also ensured in the case of a stacking of several holding parts with a high degree of certainty that the held parts are gripped sufficiently in the course of a sterilization process. Undersides of stoppers, which are received, for example, in an upper holding part, are sufficiently spaced apart from the stopper tops of the stoppers received in the holding part arranged therebelow by means of the arrangement and formation of the support walls. A sufficient spacing of the received parts when placing the holding part onto a storage surface is also favored by means of the arrangement of support walls of this type.

For this purpose, the support walls extending essentially outside of the receptacles, preferably starting from a free surface of the plate part, can have a second height, which is larger than the first height of the receptacle walls receiving and/or holding the parts. This second height of the support walls can correspond approximately—in each case preferably based on the plate plane—to 1.3 times to 2 times, further for example approximately to 1.5 to 1.6 times the first height of the receptacle wall.

In the longitudinal direction, the support wall can extend linearly or also, as further preferred, in a wave-like manner between the plurality of receptacles. A longitudinal extension of the support wall—measured along an imaginary straight line between a beginning of the support wall and an end of the support wall—can correspond up to approximately three free diameters of a receptacle, further for example approximately to 0.5 to, for example, 2 times or 3 times, further example up to 5 times the free receptacle diameter.

In an advantageous manner, several supports walls, which are preferably not connected to one another, can be formed on the holding part. These support walls can be provided so as to be distributed freely over the plate plane, wherein, in a possible design, essentially a concentration of this plurality of support walls can be provided in a central plate region of the holding part. A concentration of the support walls essentially in an outer region of holding part close to the edge can also be at hand.

According to a further solution approach, it can be provided that a receptacle wall is formed as double wall, with two concentrically circular wall parts.

The part to be received can rest on the front surfaces of the double wall or can engage over the double wall. The part can optionally also reach with one section between the walls or can be held solely in the region of the inner wall.

In the case of a step-like design of the receptacle wall, the step can have a step surface extending essentially parallel to the plate plane.

In the region of the first wall section, the receptacle wall can have several first holding formations, which protrude radially inwards. In this context, these can be column-like holding formations.

In a further design, the second wall section can also have several second holding formations, which protrude radially inward. They can likewise be formed in a column-like manner.

Starting at a wall inner surface of the first or second wall section, respectively, the first and/or second holding formations protrude freely radially inwards.

The received part, for example a stopper, can be supported point by point on the first and/or second holding formations by means of an essentially tangential abutment. In the described design, the circumferential abutment or support surface, respectively, of the part in the receptacle is further reduced by means of the arrangement of the first and second holding formations.

In a further development, the first holding formations can thereby be formed so as to be circumferentially offset from the second holding formations. In this context—with regard to a perpendicular projection of the first and second holding formations into the plate plane—this can be a circumferential offset by approximately 15 to 45 degrees, further for example approximately 30 degrees.

Three or more first or three or more second holding formations, respectively, can furthermore be provided so as to be distributed evenly viewed over the circumference, for example four to six, further for example five holding formations. The number of first and second holding formations can be selected to be different.

In a further design, the first and/or second holding formations can have a holding surface, which is curved in a cross section. Viewed in a plane parallel to the plate plane, a circular disk-shaped cross section of the holding formations can thus result, for the essentially point by point abutment of the part to be held. A protrusion position of the holding formation, which is spherical, for example, with respect to the wall inner surface, can thereby result in this cross section.

Starting at the step surface, the first holding formations can widen conically in a direction facing away from the second wall section. A cone angle of a few angular degrees can result thereby, thus, for example, approximately 0.5 to 3 degrees, further for example approximately 1 degree to an axis perpendicular to the plate plane. As a result of the conical widening of the first holding formations, a continuous reduction of the free internal dimension of the receptacle results in the region of the first wall section, starting at the step surface. A received part, for example a stopper, can accordingly circumferentially experience a lateral support in an advantageous manner by means of a point by point abutment on the first holding formations.

Viewed over the circumference, the step surface can also have several pedestal-like elevations, whereby a partial support, viewed over the circumference, of the part to be held can be attained in the receptacle. Together with the above-described holding formations in the region of the wall sections, a comparatively small support surface of the part on the receptacle results as a whole, wherein regions, which are sensitive in particular with regard to sterilization, are exposed essentially completely.

In an advantageous manner, a pedestal-like elevation can be an extension of a first holding formation. For this purpose, the first holding formation can accordingly be extended beyond the step surface. The optionally provided conical design of the holding formation can thereby continue in the elevation. The pedestal-like elevation can further have an essentially circular disk-shaped cross section according to the holding formation.

It can be provided in a further design that, starting at the step surface, the second holding formation extends all the way to a front surface of the second wall section. The second holding formation preferably does not protrude beyond the limitation of the receptacle wall. On the contrary, a free front surface of the second holding formation can run essentially at the same plane into the front surface of the second wall section. Viewed over the entire extension length, this second holding formation can thereby further have an essentially constant cross section.

On a second plate side and/or on the first plate side, several support feet can be formed on the plate part. As far as the receptacle wall extends only on one side of the plate part, the support feet can be on the side opposite to the receptacle wall. A support of the holding part having several receptacles, preferably of identical design, is made possible via these support feet, for example on the bottom of a transport and/or packaging container and/or on a further holding part, by maintaining a spacing. The dimension of the spacing is a function of the length of the support feet perpendicular to the plate plane. A stacking of several holding parts one on top of the other is also made possible via the support feet.

A spacing of the parts held in the receptacle, for example stoppers, from the bottom of the container or from the holding part provided therebelow, respectively, is also reachable via the support feet.

The several support feet can be provided so as to be distributed over the plate plane, wherein, in a possible formation, the support feet can be of identical design. Alternatively, the support feet can also be formed so as to be different, thus for example in a stump-like or also rib-like manner. A support foot can thus further have a circular cross section-viewed parallel to the plate plane.

A first support foot can expediently be formed as an extension of a first holding formation. For this purpose, the first holding formation is extended downwards beyond the respective end of the first wall section. In order to form support feet, all first holding formations can be extended in this way. However, only individual first holding formations can alternatively also form support feet in the above-described way.

These support feet can be designed to interact with parts received in the receptacles of a holding part arranged below in a stacked arrangement of several holding parts.

In a further design, the first wall section can be interrupted in circumferential direction by first free spaces. These free spaces can be limited in the circumferential direction and in the direction to the plate plane. Between the neighboring wall sections, the free spaces are not limited in a direction vertical to the plate plane. This means in a direction of the extension of the wall sections, usual a vertical extension.

The second wall section can also be interrupted around in circumferential direction by second free spaces. These second free spaces can be limited in the circumferential direction and in the direction towards the plate plane. Between neighboring wall sections, their free spaces are also not limited in a direction perpendicular to the plate plane seen from the plate plane.

If the front surface of the receptacle wall has several protrusions distributed over the circumference, in a further development they can be adapted in a radial direction to a thickness of the receptacle wall-based on a circular ring-shaped receptacle wall. The thickness of the receptacle wall thereby preferably also is given in the radial direction.

A protrusion of this type can accordingly be limited radially on the inside as well as radially on the outside by means of an axial extension of the receptacle wall, which has an outer surface and an inner surface.

A protrusion can thereby be designed in such a way that it slopes towards the outer surface of the receptacle wall. This can be attained, for example, by means of the formation of an inclined surface, further for example by means of the formation of a bezel or a rounding.

The protrusion can also merge into the inner surface and/or into the outer surface in the same plane. For example, the inner surface of the receptacle wall can thus simultaneously form the radial inner surface of the protrusion, while, for example, the protrusion can slope towards the outer surface.

According to a possible design, the circumferential extension of the protrusion can correspond approximately to the thickness of the receptacle wall. In a further design, the circumferential extension can correspond, for example, to approximately 0.8 times to approximately 2 times, further for example to approximately 1 time to 1.5 times the thickness.

On its second plate side lying opposite the receptacle wall, the plate part can also have one or several second support feet. With respect to a central axis, a second support foot can be formed so as to be offset radially outwards in relation to an imaginary continuation of the receptacle all the way to the opposite side. An arrangement of several support feet can thus be at hand, which, in the case of an exemplary stacking of several holding parts of identical design outside of the receptacles there, are supported, for example, directly on the plate part.

In addition to the support walls, one or several support posts can also be provided, which can also have the second height. A homogenous distribution of support walls and support posts can be at hand thereby. It may be preferable, however, that the support walls are provided essentially in a central region of the holding part and the support posts essentially in an outer region close to the edge. The reverse arrangement is also possible in this context, accordingly with support posts essentially in the central region of the holding part and support walls essentially in the region close to the edge.

Due to preferably identical second heights of support walls and support posts, a common support plane defined by the free front surfaces of the support walls and support posts can result.

The support posts can additionally further also be formed by means of the described support feet.

In the case of a formation of a receptacle with double wall, the wall parts can be connected on their ends, which are spaced apart from the plate plane, by means of a front surface in a further development. For example, a U-shaped design can result in the connecting region of the wall parts in a cross section in a plane perpendicular to the plate plane. The front surface can extend in parallel alignment to the plate plane.

The front surface can also have openings. These openings can extend solely in the region between the adjoining wall parts.

A plurality of openings, preferably openings of identical design and spaced apart evenly from one another, can be provided in the front surface, thus, for example, three to seven, further, for example five.

In a possible design, the openings can be formed so as to be elongated. In the circumferential direction of the double wall, the openings can in each case extend, for example, over an angle of approximately 30 to approximately 60 degrees, further for example approximately 45 degrees.

In a further development, a wall part of the double wall can merge into a protrusion on a second plate side of the plate part lying opposite the receptacle. This can thereby be a protrusion of the above-described type, for example for supporting a cap to be held.

In a possible design, the radially inner wall part can merge into the protrusion.

The front surface of the receptacle wall can be formed in a stepped manner in the circumferential direction. Accordingly, front surface planes result, which are spaced apart differently from the plate plane. For example, two front surface planes can thus be at hand, with a first front surface plane closer to the plate plane and a second front surface plane further away from the plate plane. A support protrusion protruding beyond the first front surface plane can be formed by means of this plane offset.

In a possible design, the second front surface plane can assume a distance from the plate plane, which corresponds to the second height, for example of the support walls and/or of the support posts.

Several support protrusions can be formed over the circumference. For example, two or three support protrusions, which are spaced apart from one another in the circumferential direction, can thus be provided.

The support protrusions of at least the same receptacle can also be of identical design.

In a possible design, the front surface in the second front surface plane can be formed so as to be closed. In this case, the openings result solely between the support protrusions in the region of the front surfaces of the first front surface plane. However, openings can alternatively be provided in the first as well as in the second end surface plane, further alternatively only in the second front surface plane, while the front surface of the first front surface plane can optionally not have any openings.

With regard to the disclosure, the above- and below-specified ranges or value ranges, respectively, or multiple ranges also include all intermediate values, in particular in 1/10 steps of the respective dimension, optionally also without dimension. For example, the specification 15 to 45 degrees also includes the disclosure of 15.1 to 45 degrees, 15 to 44.9 degrees, 15.1 to 44.9 degrees, etc., the disclosure of 3 times to 5 times also includes the disclosure of 3.1 to 5 times, 3 to 4.9 times, 3.1 to 4.9 times, etc., On the one hand, this disclosure can serve the purpose of limiting a mentioned range limit from below and/or above, but alternatively or additionally also for the purpose of disclosing one or several singular values from a respective specified range.

### BRIEF DESCRIPTION OF THE DRAWINGS

The invention is described below on the basis of the enclosed drawings, which only represent exemplary embodiments, however. A part, which is described only based on one of the exemplary embodiments and which is not replaced by another part in the case of a further exemplary embodiment due to the special features emphasized there, is thus also described for this further exemplary embodiment as at least possible available part. In the drawing:

FIG. 1 shows a plate-like holding part, relating to a first embodiment, in perspective illustration;

FIG. 2 shows the enlarged region II from FIG. 1 with two receptacles;

FIG. 3 shows the top view onto a receptacle according to arrow Ill in FIG. 2;

FIG. 4 shows the bottom view of the receptacle according to arrow IV in FIG. 2;

FIG. 5A shows the section along the line V-V in FIG. 3, in the case of a stacked arrangement of several holding parts of the first embodiment shown in dash-dotted line type;

FIG. 5B shows the enlarged region Va in FIG. 5A;

FIG. 6 shows the enlarged region VI in FIG. 5A;

FIG. 7 shows an illustration corresponding to FIG. 2, but when holding a part in the form of a stopper in one of the receptacle;

FIG. 8 shows the top view according to arrow VIII in FIG. 7;

FIG. 9 shows the section through the plane IX in FIG. 7, in the case of a stacked arrangement of several holding parts of the first embodiment shown in dash-dotted line type;

FIG. 10 shows the section through the plane X in FIG. 2;

FIG. 11 shows a sectional illustration according to FIG. 10, but in a changed alignment of the perspective;

FIG. 12 shows a schematic top view onto the holding part, with a cutout enlargement;

FIG. 13 shows the section along the line XIII-XIII in FIG. 12;

FIG. 14 shows a perspective illustration according to FIG. 11, relating to a second embodiment;

FIG. 15 shows a sectional illustration according to FIG. 9, relating to the embodiment according to FIG. 14;

FIG. 16 shows the holding part of the second embodiment in a schematic top view;

FIG. 17 shows a stacked arrangement of several holding parts, relating to a third embodiment, in a transport vessel in perspective illustration;

FIG. 18 shows the section through the plane XVIII in FIG. 17;

FIG. 19A shows the enlarged region XIX in FIG. 18;

FIG. 19B shows an illustration according to FIG. 19A, but with an alternative design;

FIG. 20 shows the region XX in FIG. 17, but without part held in the receptacle;

FIG. 21 shows the bottom view according to arrow XXI in FIG. 20;

FIG. 22A shows an illustration corresponding to FIG. 20, but with part in the form of a cap held in the receptacle;

FIG. 22B shows the region as shown in FIG. 20, but relating to a further embodiment of the receptacles;

FIG. 23 shows an illustration essentially corresponding to FIG. 20, relating to a fourth embodiment;

FIG. 24 shows the top view according to arrow XXIV in FIG. 23;

FIG. 25 shows the enlarged region XXV in FIG. 23;

FIG. 26 shows an illustration corresponding to FIG. 25, relating to an alternative design;

FIG. 27 shows the bottom view according to arrow XXVII in FIG. 23;

FIG. 28 shows the section along the line XXVIII-XXVIII in FIG. 24, in the case of a stacked arrangement of several holding parts of the fourth embodiment, represented in dash-dotted line type;

FIG. 29 shows an illustration corresponding to FIG. 23, when holding a cap on the receptacle;

FIG. 30 shows the top view according to arrow XXX in FIG. 29;

FIG. 31 shows the section along the line XXXI-XXXI in FIG. 30, in the case of a stacked arrangement of several holding parts of the fourth embodiment represented in dash-dotted line type;

FIG. 32 shows the holding part of the fourth embodiment in a schematic top view, with a detail enlargement;

FIG. 33 shows a perspective bottom view of a holding part in a fifth embodiment;

FIG. 34 shows the top view according to arrow XXXIV in FIG. 33;

FIG. 35 shows the enlarged region XXXV in FIG. 34;

FIG. 36 shows the section along the line XXXVI-XXXVI in FIG. 35;

FIG. 37 shows an illustration corresponding to FIG. 33, relating to a sixth embodiment;

FIG. 38 shows the top view according to arrow XXXVIII in FIG. 37;

FIG. 39 shows the enlarged region XXXIX in FIG. 38;

FIG. 40 shows the section along the line XL-XL in FIG. 39;

FIG. 41 shows a further illustration corresponding to FIG. 33, relating to a seventh embodiment;

FIG. 42 shows the top view according to arrow XLII in FIG. 41;

FIG. 43 shows the enlarged region XLIII in FIG. 42;

FIG. 44 shows the section along the line XLIV-XLIV in FIG. 43;

FIG. 45 shows a further illustration corresponding to FIG. 33, relating to an eighth embodiment;

FIG. 46 shows the top view according to arrow XLVI in FIG. 45;

FIG. 47 shows the enlarged region XLVII in FIG. 46;

FIG. 48 shows the section along the line XLVIII-XLVIII in FIG. 47;

FIG. 49 shows a further illustration corresponding to FIG. 1, relating to a ninth embodiment;

FIG. 50 shows the enlarged region L from FIG. 49 with two receptacles;

FIG. 51 shows the top view onto a receptacle according to arrow LI in FIG. 50;

FIG. 52 shows the bottom view of the receptacle according to arrow LII in FIG. 50;

FIG. 53 shows an enlarged area as shown in FIG. 6, but relating to the embodiment shown in FIG. 49;

FIG. 54 shows an illustration corresponding to FIG. 50, but when holding a part in the form of a stopper in one of the receptacle;

FIG. 55 shows the top view according to arrow LV in FIG. 54;

FIG. 56 shows the section through the plane LVI in FIG. 54, in the case of a stacked arrangement of several holding parts of the ninth embodiment shown in dash-dotted line type;

FIG. 57A shows the section through the plane LVII in FIG. 50;

FIG. 57B shows the enlarged region LVIIa in FIG. 57A;

FIG. 58 shows a sectional illustration according to FIG. 57A, but in a changed alignment of the perspective;

FIG. 59 shows a schematic top view onto the holding part of the ninth embodiment;

FIG. 60 shows the enlarged region LX from FIG. 59 with a free-standing insertion receptacle;

FIG. 61 shows the section through the plane LXI in FIG. 60;

FIG. 62 shows the bottom view of the holding part according to arrow LXII in FIG. 61;

FIG. 63 shows an enlarged area as shown in FIG. 60, but relating to a further embodiment of the insertion receptacle; and

FIG. 64 shows a schematic perspective illustration to describe the stacked arrangement of holding parts for receiving different parts.

### DESCRIPTION OF THE EMBODIMENTS

What is illustrated and described is a plate-like holding part 1 for holding parts T, such as stoppers 2, for example made of an elastomer material, or caps 3, for example aluminum crimp caps, for closing pharmaceutical vessels, preferably pre-sterilized vessels 4, such as, filling cartridges or vials, for example, are optionally supplied to them automatically.

With regard to the holding part, it can be a tray or nest structure.

As illustrated schematically, for example, in FIG. 64, plate-like holding parts 1 for the same parts T, for example stoppers 2 or caps 3 or also vessels 4, can be capable of being arranged in a stacked arrangement in a transport vessel 5. After a sterilization was carried out, the stack can be received together with the transport vessel 5 in a sterile manner, for example in a bag 47, and can be supplied for further transport or for further machine processing.

The holding part 1 has a plate part 6 with a plate plane E. The plate part 6 essentially specifies the width b and length a of the holding part 1. The length a can thereby correspond approximately to 1.05 to 1.5 times, further for example approximately 1.3 times the width b. For example, a length of 250 to 350 mm can be given, with an exemplary width of 150 to 300 mm. The first and second plate sides 10 and 22 preferably run parallel to one another. The plate plane E can be at hand, for example, centrally between the first and second plate sides 10 and 22, viewed in the thickness direction of the plate part 6.

The holding part 1 further has a plurality of receptacles 7 for holding the parts T, which receptacles 7, per se, extend perpendicular to the plate plane E. As illustrated, each receptacle 7 can have a circular cylindrical receptacle wall 8, which runs so as to be aligned essentially concentrically to a central axis x aligned perpendicular to the plate plane E and which limits a central receptacle opening 9, which also breaks through the plate part 6.

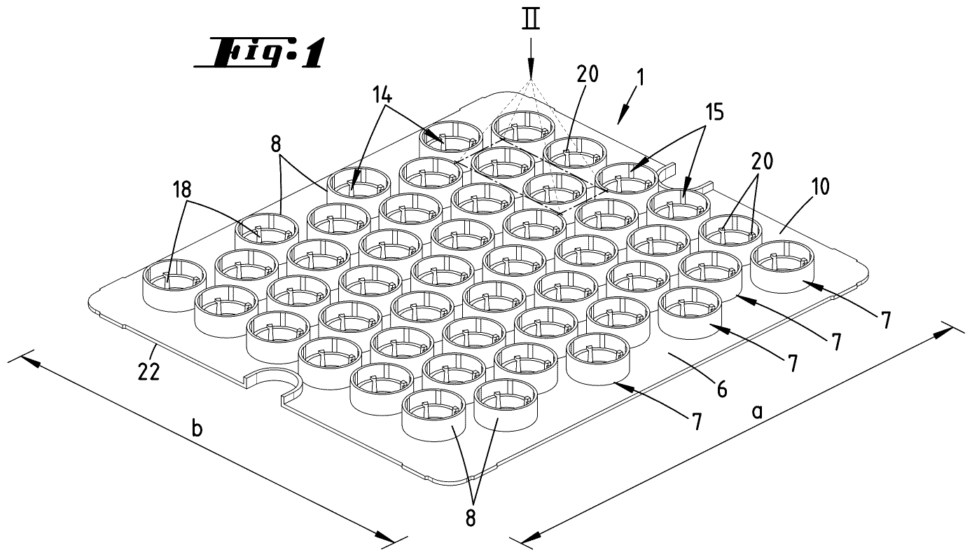

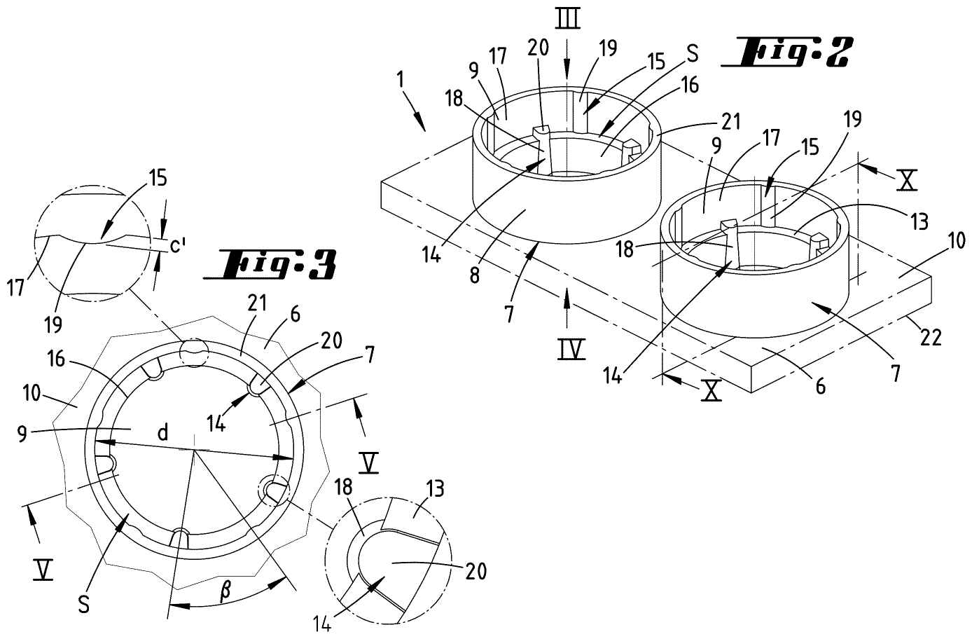

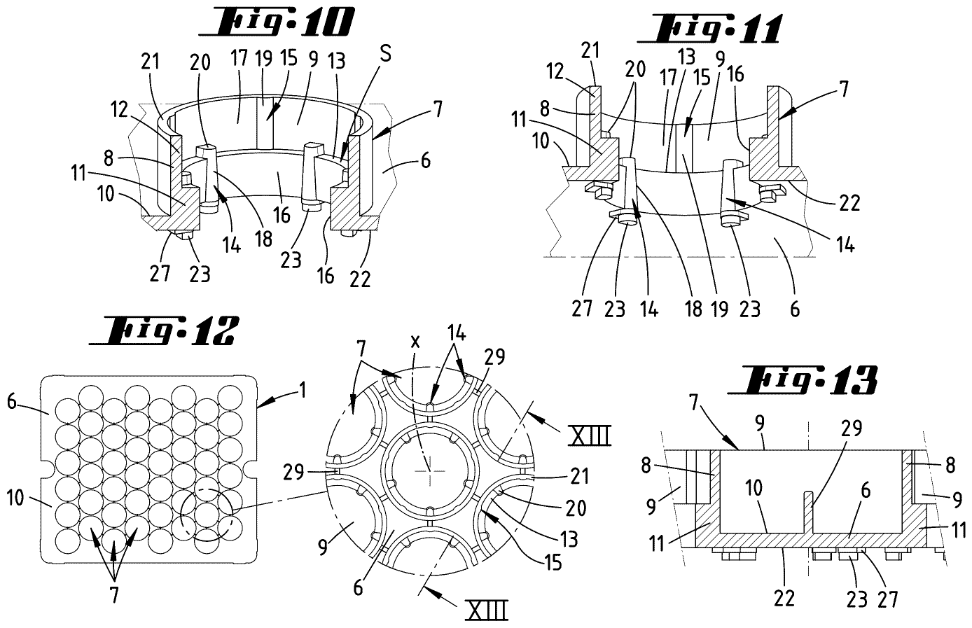

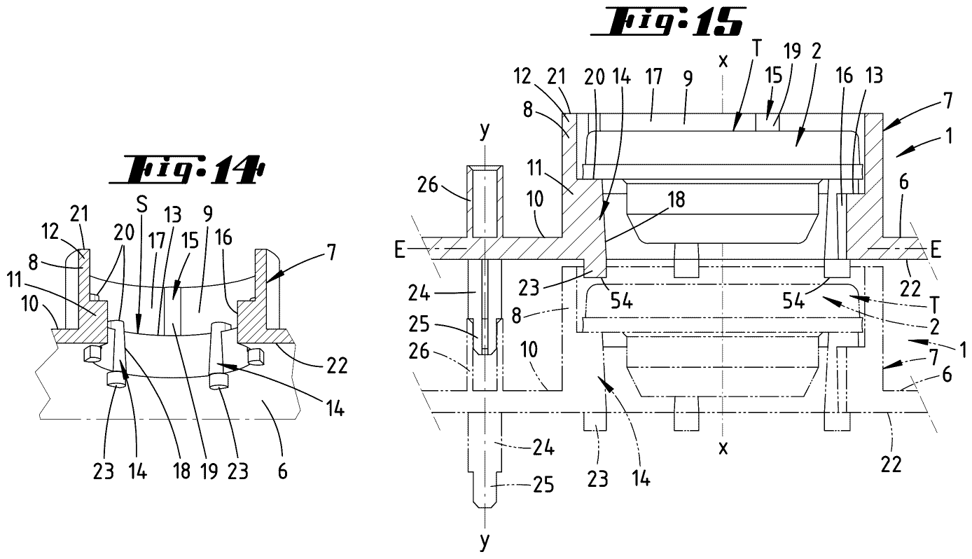

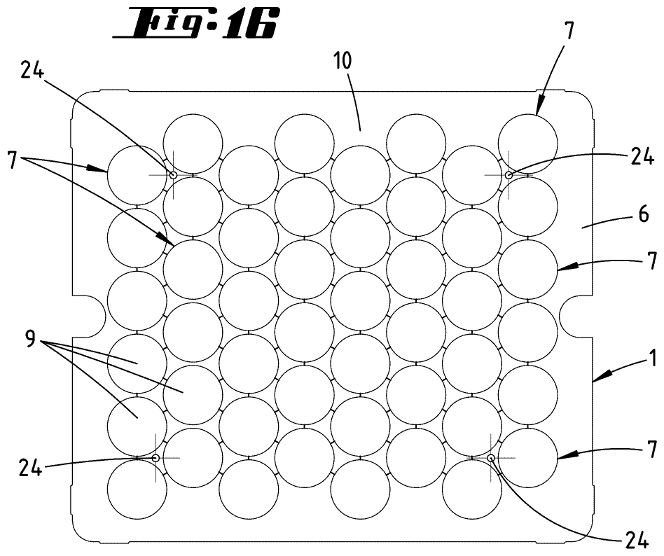

The receptacle wall 8 of the first and second embodiments illustrated in FIGS. 1 to 16 extends at least essentially completely starting at a first plate side 10 of the plate part 6 pointing upwards in the usual alignment of the holding part 1 according to FIG. 1.

Viewed in alignment of the central axis x, the receptacle wall 8 of this embodiment is formed in a stepped manner. This results in a first wall section 11 closer to the plate plane E and a second wall section 12 further away from the plate plane E. The first wall section 11 merges into the second wall section 12 via a step S with a step surface 13, which runs essentially parallel to the plate plane E.

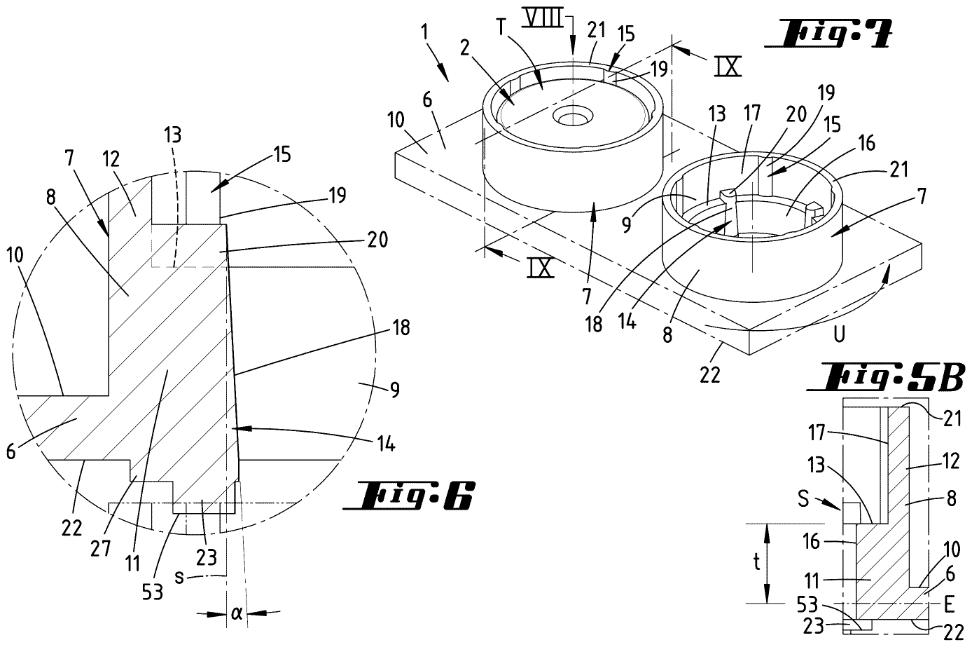

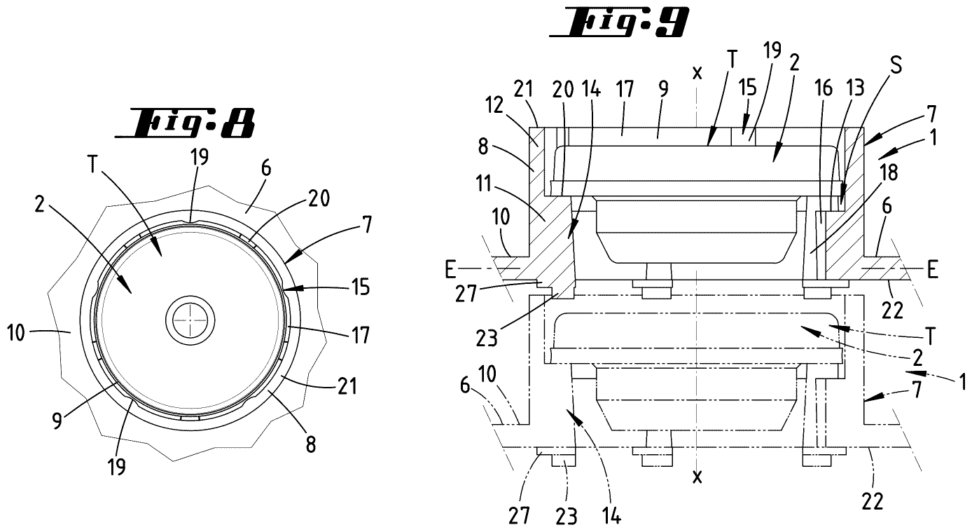

As can also be seen in particular from the enlargement in FIG. 5B, the first wall section 11 extends in the axial direction from the (second) front surface 53 of the first supporting foot 23 and/or from the second plate side 22, passing through the plate plane E, to the step S or the step surface 13 formed by the step S. The step surface 13 has an axial distance t from the plate plane E.

The second wall section 12 extends in the axial direction from the step surface 13 to a first front surface 21, starting at a distance t from the plate plane E.

A free inner diameter d—measured in the region of the second wall section 12—can result in the region of the step surface 13, which can correspond, for example, approximately to 1.1 to 1.3 times, further for example approximately to 1.1 times the free diameter d′ of the first wall section 11.

The distance t between the step S and the plate plane E can correspond to approximately one tenth to approximately one third, furthermore for example approximately one fifth of the inner diameter d of the second wall section 12. This distance t between the step S and the plate plane E can also be very small, for example 0.2 times or less of the diameter d, and possibly even close to or equal to zero. In the alternative as to a description of the possible dimension of the distance t, the distance t can be between 0 and 10 mm, preferably between 0.5 and 2.5 mm. Any in-between values are also possible, preferably in steps of 0.1 mm from the upper and/or lower value. For example, it can be between 0.1 and 10 mm, 0.0 and 9.9 mm, 0.2 and 10 mm, 0.0 and 9.8 mm and so on.

In a further design, the first wall section 11 has several first holding formations 14, which protrude radially inwards and which protrude into the receptacle opening 9. In this context, these can be like holding formations 14, which are aligned in the direction of the central axis x and which extend in a column-like manner. A radial protrusion dimension c with respect to the wall inner surface 16 of the first wall section 11 can result thereby, which protrusion dimension c can correspond, for example, approximately to 0.015 to 0.025 times the diameter d′ of the first wall section 11 (see FIG. 4).

The second wall section 12 can also have several second holding formations 15, which protrude radially inwards. They can likewise be formed in a column-like manner and can have a protrusion dimension c′ with respect to the wall inner surface 17 of the second wall section 12, which can be adapted to the protrusion dimension c of the first holding formations 14.

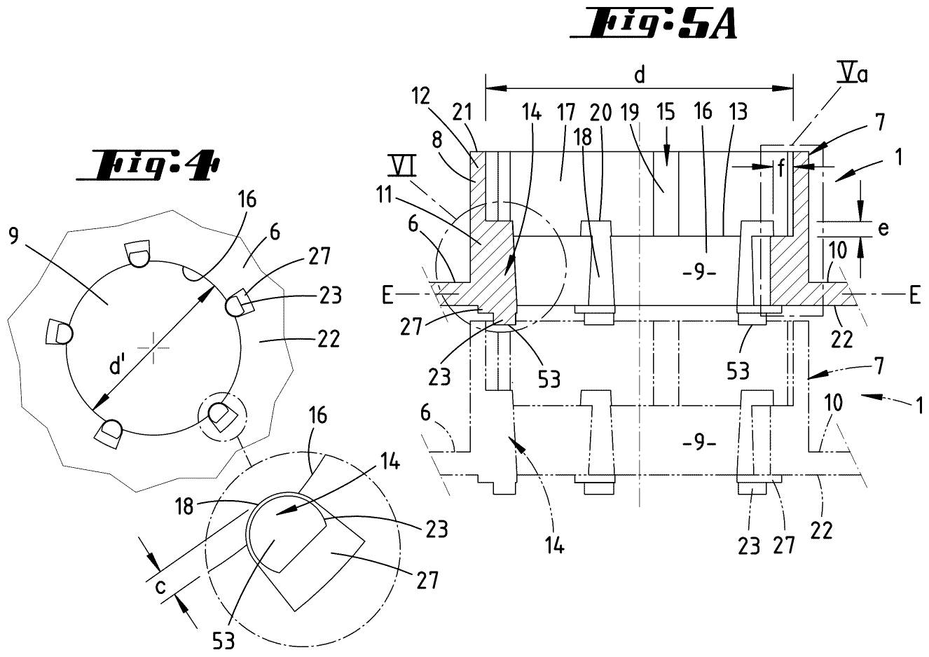

In relation to a projection of the holding formations 14 and 15 into the plate planes E, the first holding formations 14 can be formed so as to be circumferentially offset from the second holding formations 15 into the plate plane E. In this context, this can be a circumferential offset over an angle β of approximately 30 degrees (see FIG. 3).

Five first and five second holding formations 14, 15 can furthermore be provided so as to be distributed evenly viewed over the circumference.

In further design, the first and second holding formations 14, 15 can have a holding surface 18, 19, which is curved in a cross section. Viewed in a plane parallel to the plate plane E, a cross section of the holding formations 14, 15 can thus result in the form of a circular disk section. The holding formations 14, 15 can protrude spherically by the respective protrusion dimension c or c′, respectively, beyond the respective wall inner surface 16, 17.

Starting at the step surface 13, the first holding formations 14 can widen conically in a direction facing away from the second wall section 12. A cone angle α of, for example, approximately 1 degree to a perpendicular s—based on the plate plane E—can result thereby. Starting at the step surface 13 viewed in the direction of the plate part 6, a continuous reduction of the free dimension viewed transversely to the central axis x results accordingly in the region of the first holding formations 14.

Starting at the step surface 13, the second holding formations 15 extend all the way to the first front surface 21 of the second wall section 12, which is directed upwards in the usual alignment of the holding part 1 according to FIG. 1. The free front surfaces of the second holding formations 15 can accordingly run into the first front surface 21 of the second wall section 12 essentially in the same plane. These second holding formations 15 can thereby further have an essentially constant cross section, in particular circular disk cross section, over the entire extension length viewed in the direction of the central axis x.

Viewed over the circumference, the step surface 13 can also have several pedestal-like elevations 20. These pedestal-like elevations 20 can each be formed in an advantageous manner as an extension of a first holding formation 14, which goes beyond the step surface 13 in the direction of the central axis x. For this purpose, the first holding formation 14 is accordingly extended beyond the step surface 13, for the formation of the elevation 20. The optionally provided conical design of the first holding formation 14 can thereby also continue in the elevation 20. The pedestal-like elevation 20 can further have an essentially circular disk-shaped cross section according to the first holding formation 14.

Viewed in the direction of the central axis x, the elevation 20 can extend beyond the step surface 13 with a measure e, which measure e can correspond, for example, approximately to 0.8 to 1.5 times the radial dimension f of the step surface 13 between the wall inner surfaces 16 and 17.

In a further design, several first support feet 23 can be formed on the plate part 6 on a second plate side 22 lying opposite to the receptacle wall 8. A support of the holding part 1, for example on the bottom of a transport and/or packaging vessel 5 and/or in the case of a stacked arrangement on a further holding part 1, can be made possible via these first support feet 23, by maintaining a spacing.

A first support foot 23 can be formed as an extension of a first holding formation 14, which protrudes beyond the second plate side 22. For this purpose, the first holding formation 14 is extended downwards beyond the respective end of the first wall section 11. All first holding formations 14 can be extended in this way to form first support feet 23. However, only individual first holding formations 14 can alternatively also form first support feet 23 in the above-described manner.

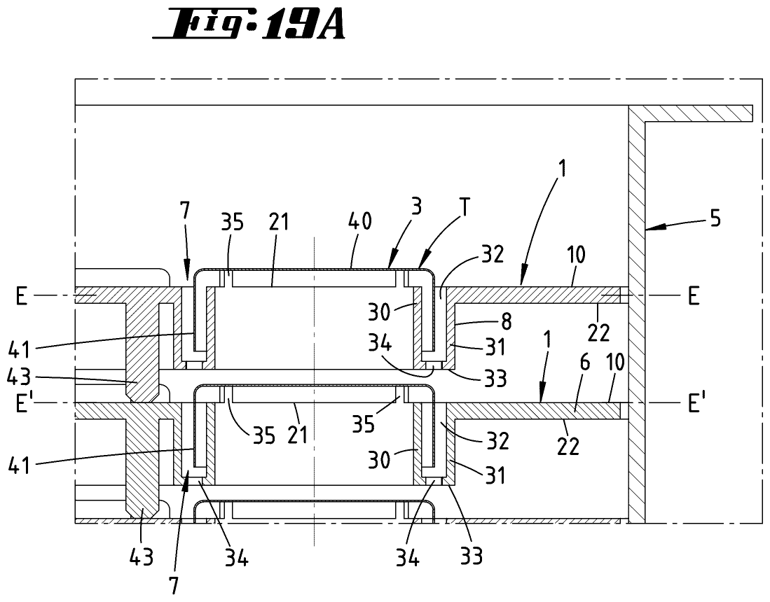

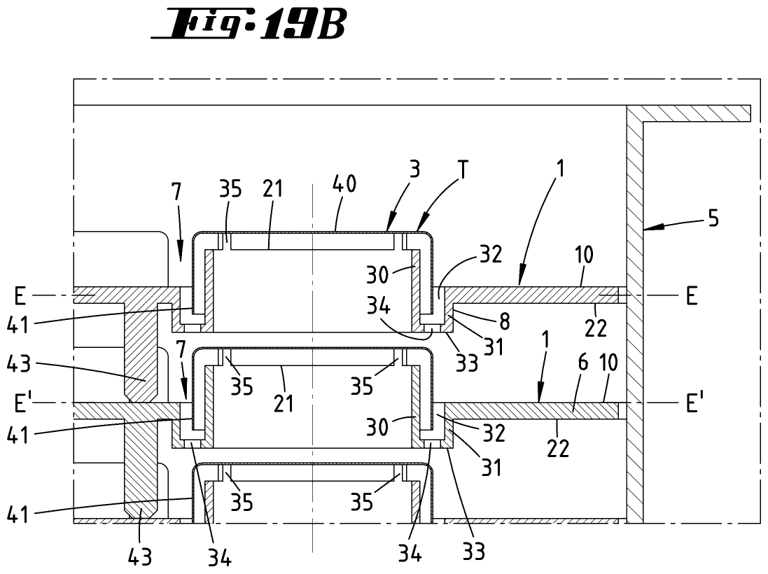

The first support feet 23 can be formed solely by means of such an extension of the first holding formations 14 (see second embodiment in FIGS. 14 to 16). In addition to these first support feet 23, which are assigned essentially directly to the receptacles 7, further, preferably pin-like second support feet 24 can be provided in this case, which can be provided in a region of the holding part 1 close to the edge according to the schematic illustration in FIG. 16.

These second separate support feet 24 are also formed on the second plate side 22 lying opposite the receptacle wall 8. In relation to a central axis y, a second support foot 24 can thereby be formed so as to be offset radially outwards in relation to an imaginary continuation of the receptacle 7 or of the receptacle wall 8, respectively, all the way to the opposite (second) plate side 22. An arrangement of several second support feet 24 can thus be at hand, which, in the case of an exemplary stacking of several holding parts 1 of identical design outside of the receptacles 7 there, are supported on the plate part 6.

The second support feet 24 can have a profiled insertion section 25 in the region of their free end.

An insertion receptacle 26 can be formed in the axial extension of the second support foot 24 on the top side of the plate part 6, assigned accordingly to the first plate side 10, for the plug-in reception of an insertion section 25 of a holding part 1, which is further provided in the stacked arrangement above (see FIG. 15).

The axial lengths of the second support feet 24 and of the insertion receptacles 26 can be adapted to the axial extensions of the receptacle walls 8 of the holding part 1.

A spacing of the parts T held in the receptacles, for example stoppers 2, to the bottom 28 of the transport vessel 5 or to the holding part 1 further provided therebelow, respectively, is also ensured via the support feet.

The second several support feet 24 can be provided so as to be distributed over the plate plane E, wherein the second support feet 24 can be of identical design.

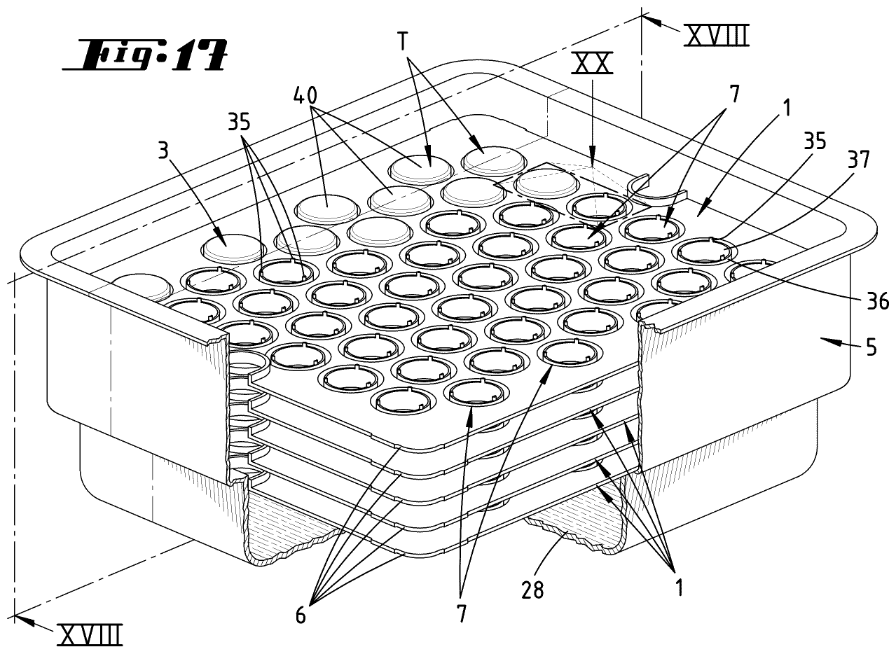

The first support feet 23 formed essentially by means of extension of the first holding formations 14 engage below the second plate side 22 in a region close to the edge of the receptacle opening 9 (see FIGS. 11 and 14).

In the first exemplary embodiment shown in FIGS. 1 to 13, but also not limited to this, the first support foot 23 can be designed in a step-like manner, with a step section 27, which is interconnected essentially between the second plate side 22 and the first support foot 23 and which forms an enlarged surface for engaging below to the second plate side 22. In the stacked arrangement according to FIGS. 9 and 15, a first support foot 23 of the upper holding part 1 can engage into the receptacle opening 9 of the lower holding part 1 and can hold parts T, for example a stopper 2, in place during transportation by working together with the top surface of such part T.

When holding a part T, in particular a stopper 2, in the receptacle 7, an essentially point by point support of the part T can result in an advantageous manner in the region of the step surface 13 on the elevations 20 provided there. The contact surface of the part T, which is to be received and held, is so small that a sufficient sterilization can be carried out, for example in the receiving position in the holding part 1.

The received part T can also be supported only point by point on the first and/or second holding formations 14, 15 by means of an essentially tangential abutment in the circumferential direction U on the first and/or second holding formations 14, 15.

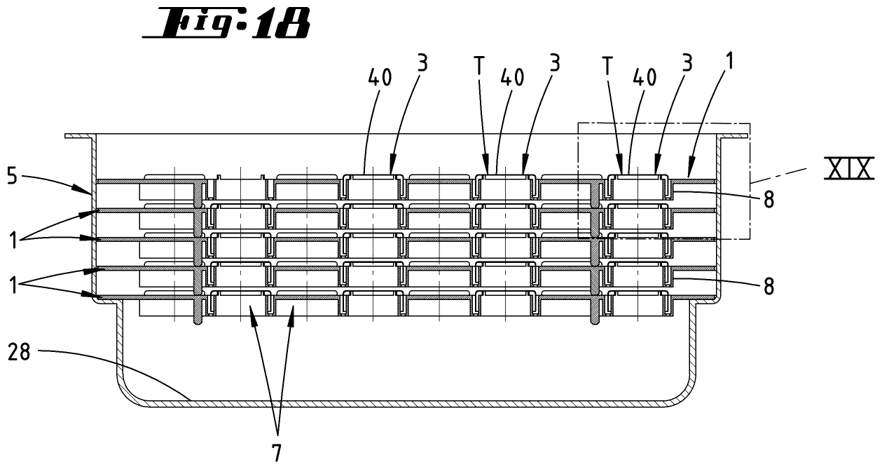

As can further be seen from FIGS. 12 and 13, stabilizing connecting bridges 29, which connect the receptacle walls 8 to one another, can be provided, assigned to the first plate side 10 between the receptacle walls 8 of the receptacles 7. The connecting bridges 29 can run so as to be oriented along a radial relating to the central axis x. It is also possible to design the holding part 1 without such connecting bridges 29.

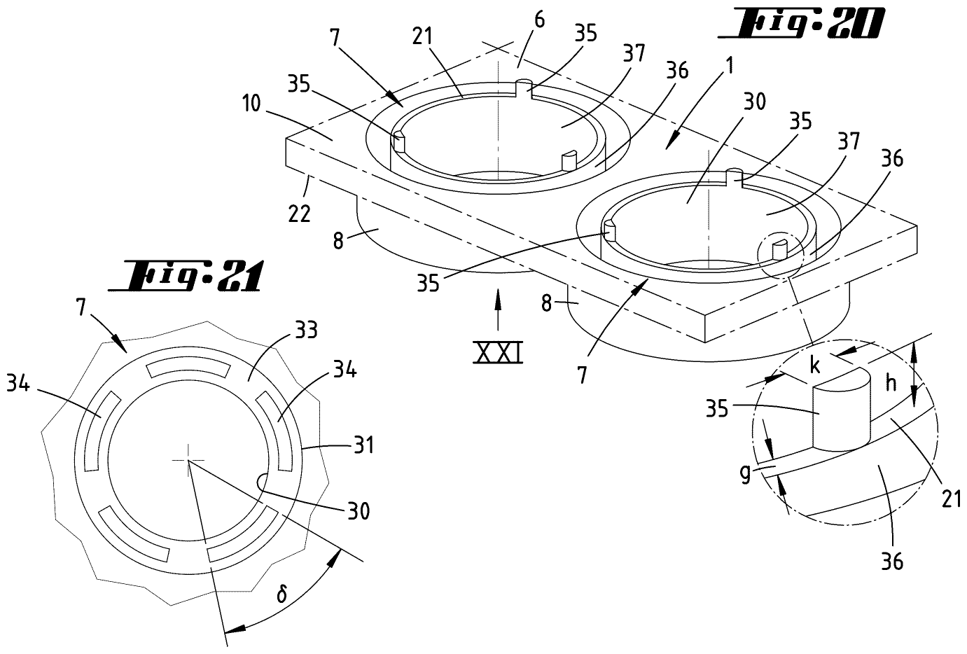

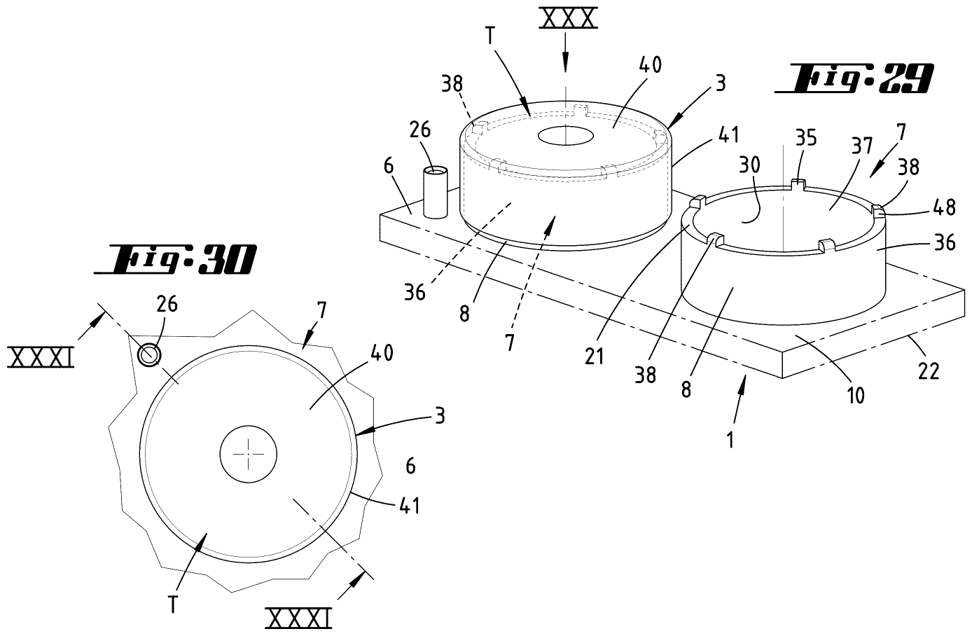

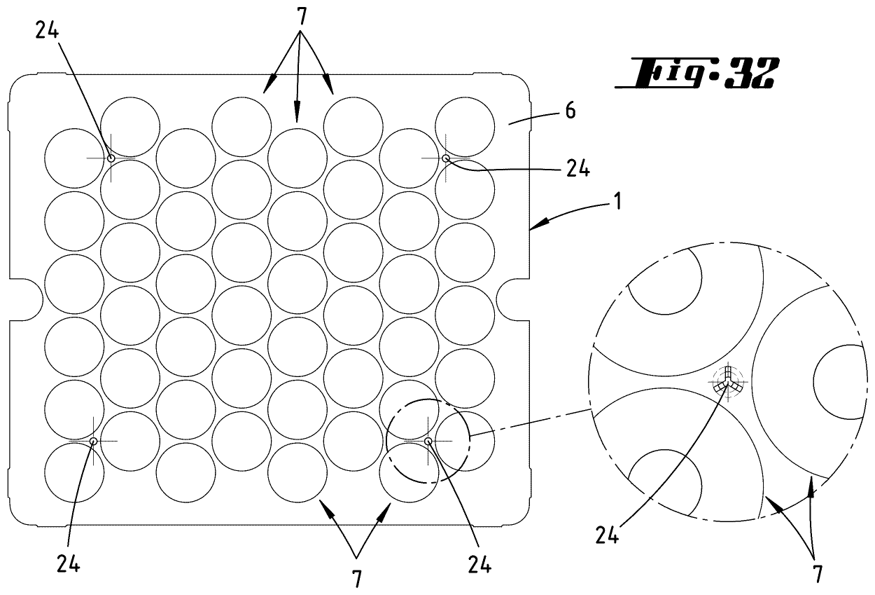

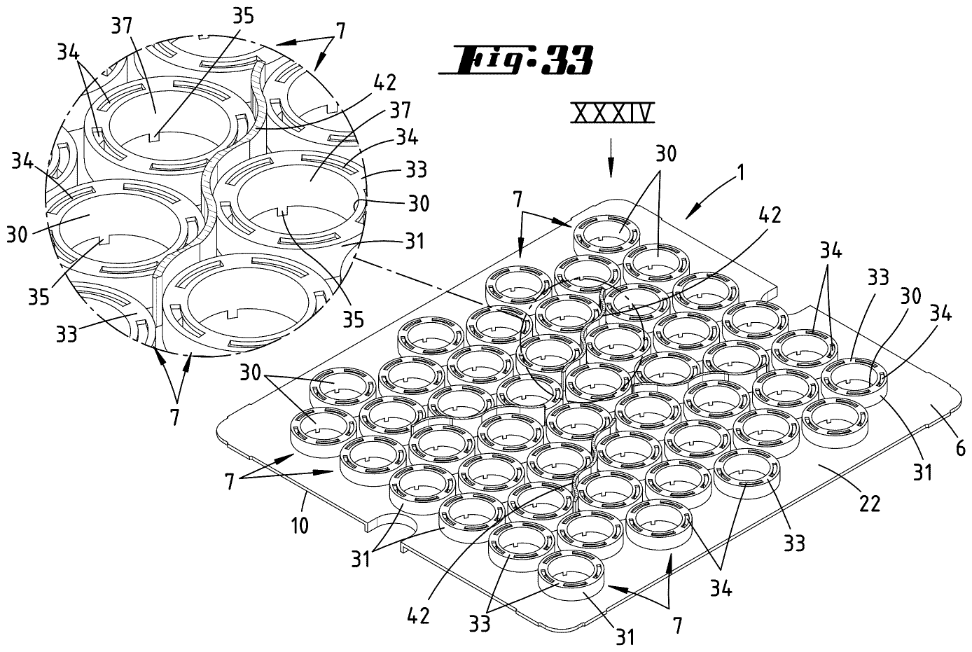

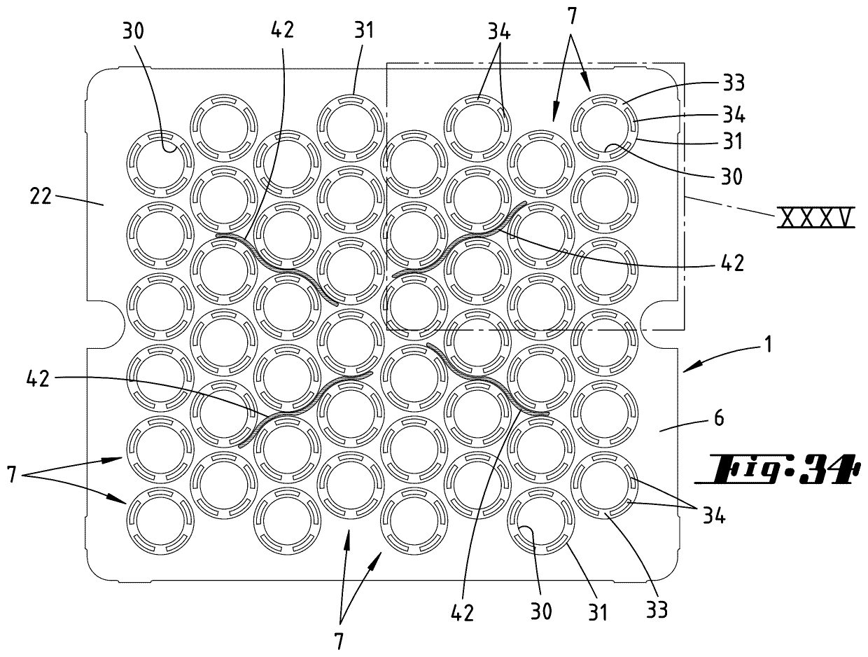

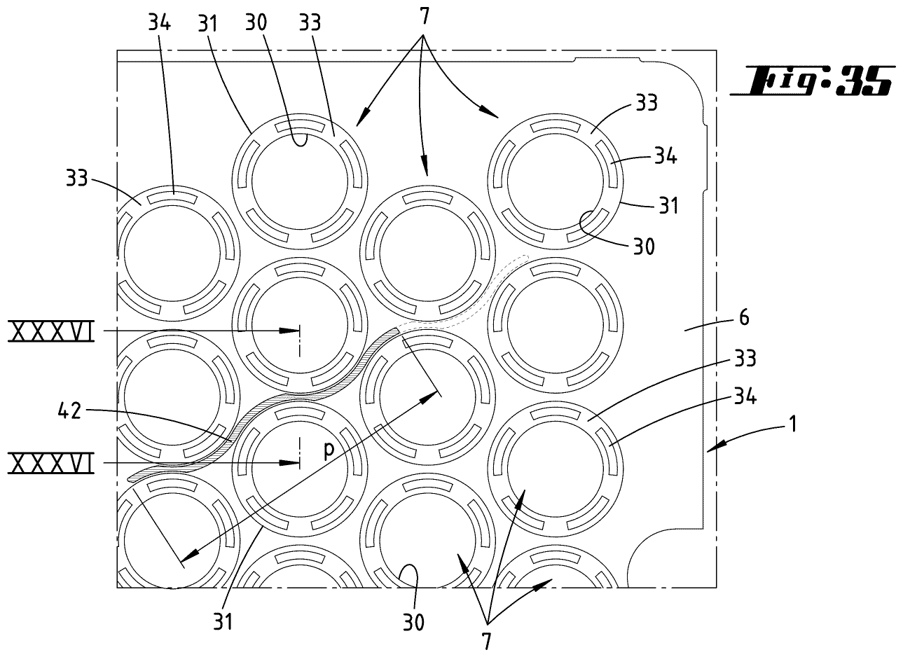

The embodiments three and four illustrated in FIGS. 17 to 32 show holding parts 1 for holding parts T, preferably in the form of caps 3.

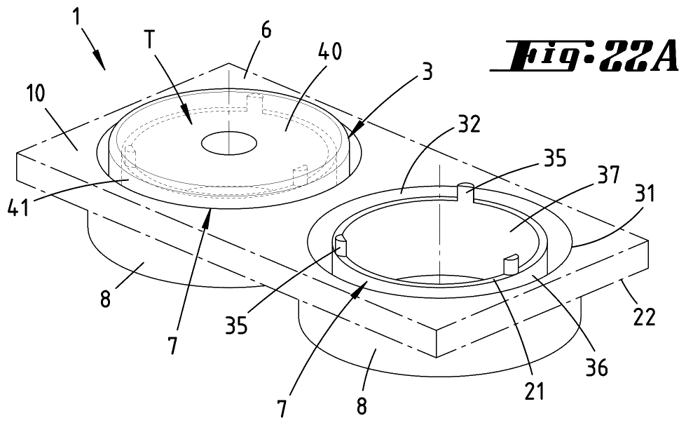

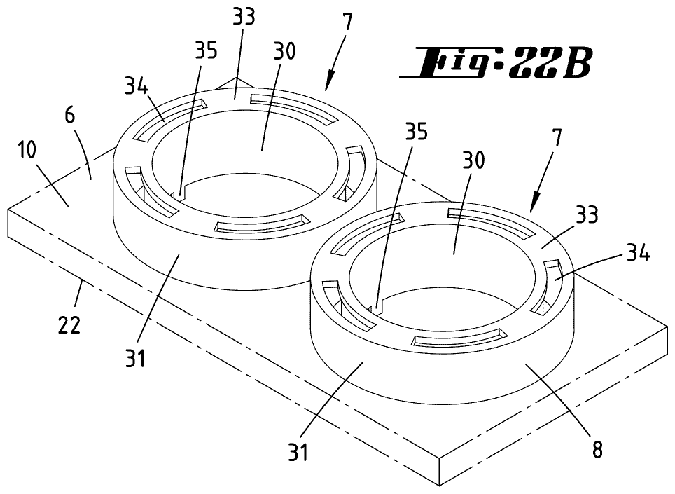

As can be seen in particular from FIG. 19A, the receptacle wall 8 extends essentially below the plate plane E in the third embodiment.

FIG. 19B shows an alternative design in which the receptacle wall 8 extends partly above and partly below the plate plane E. The basic design of the receptacle 7 is preferably the same as the embodiment shown in FIG. 19A.

The receptacle wall 8 can be formed as double wall, with two concentrically circular wall parts, a radially inner wall part 30 and a radially outer wall part 31. A circular ring-shaped slit 32 results between the wall parts 30 and 31. In the case of the exemplary embodiment illustrated in FIGS. 17 to 22B, the wall parts 30 and 31 extend on the underside of the plate part 6 essentially starting at the second plate side 22.

In the embodiment example shown in FIG. 19B, the inner wall part 30 extends from an area below the plate plane E, passing through the plate plane E to an area above the plate plane E, while the radially outer wall part 31 preferably extends below the plate plane E alone. The first front surface 21 of the inner wall part 30 extends at an axial distance above the first plate side 10. The front surface 33, which also limits the outer wall part 31 in the axial direction, extends at an axial distance below the second plate side 22.

On their ends spaced apart from the plate plane E, the wall parts 30 and 31 can be connected by means of a front surface 33. In a cross section according to FIG. 19A, an essentially U-shaped design can result in the connecting region of the wall parts 30 and 31 in a plane perpendicular to the plate plane E, whereby the front surface 33 can extend in parallel alignment to the plate plane E.

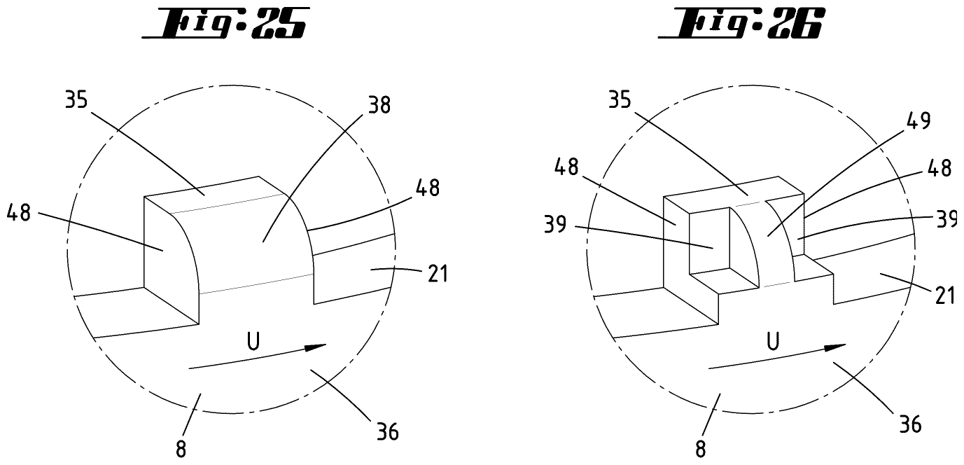

The double-walled formation of the receptacle wall 8 can also be at hand in the case of a receptacle 7, which extends essentially above the plate plane E. In this case, the front surface 33 connecting the wall parts 30 and 31 points upwards and assumes an axial distance from the first plate side 10 (see FIG. 22B).

The receptacle wall 8, also as double wall, can furthermore generally be arranged so that it passes through the plate plane E and accordingly extends partly above and partly below the plate plane E.

The front surface 33 of the double wall can have openings 34, which are elongated in the circumferential direction U. These openings 34 can be provided solely in the region between the adjacent wall parts 30 and 31, without the openings 34 extending into the region of the wall parts 30 and 31. A plurality of openings 34 of preferably identical design can be provided thereby. The openings 34 can furthermore be evenly spaced apart from one another in the circumferential direction U.

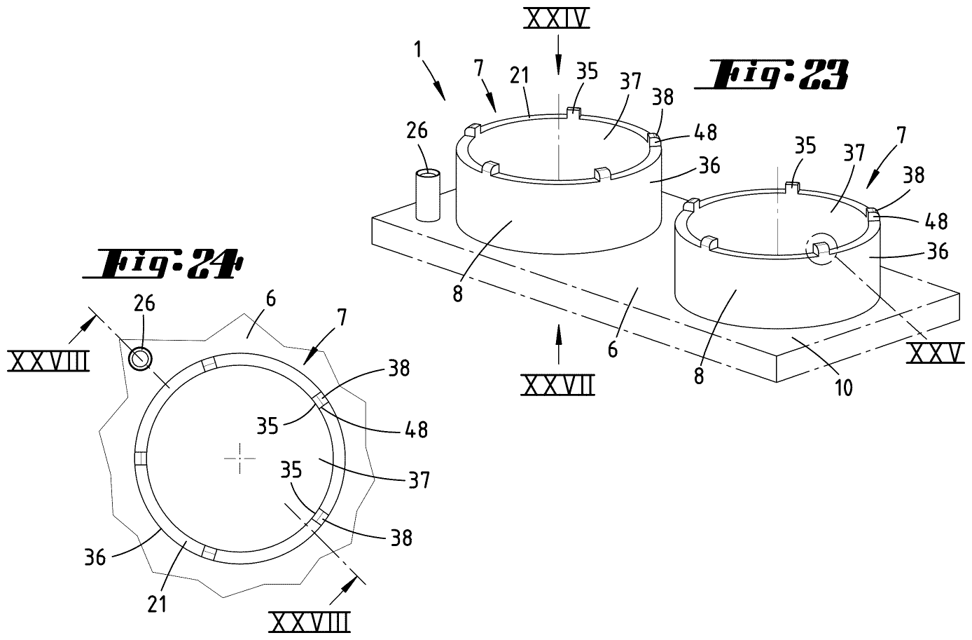

Viewed in the circumferential direction of the double wall, five such openings 34 can thus be provided, which each extend over an angle δ of approximately 45 degrees (see FIG. 21).

Several protrusions 35 can be formed, distributed over the circumference, on the first front surface 21 of the receptacle wall 8, which point upwards in the usual use position and which runs essentially in a plate-parallel manner, or, respected to the double-walled formation, on the first front surface 21 of the radially inner wall part 30, respectively.

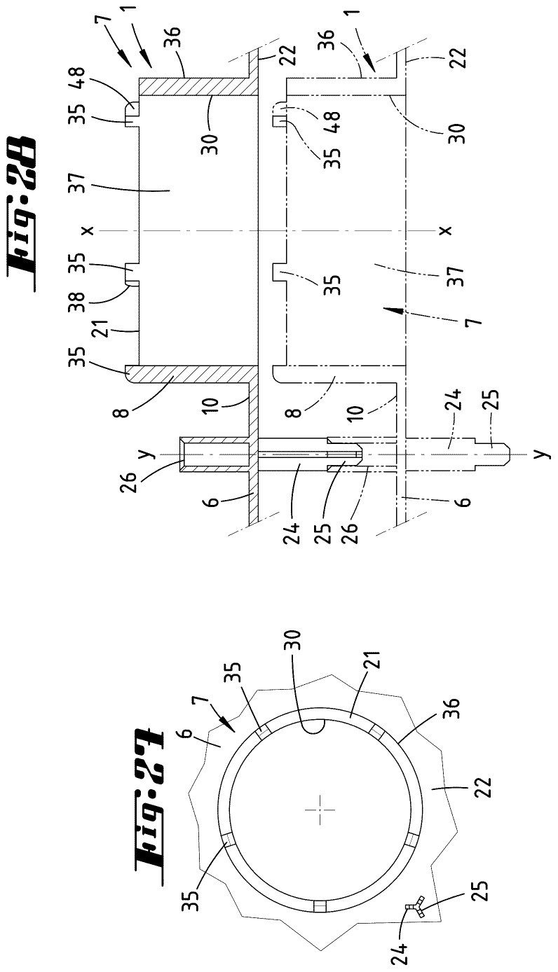

According to the embodiment illustrated in FIGS. 17 to 22, three such protrusions 35 can be provided, distributed evenly over the circumference. The embodiment in FIGS. 23 to 32 shows a receptacle wall 8 with five protrusions 35.

The protrusions 35 can be adapted in the radial direction to a thickness g of the receptacle wall 8 or of the radially inner wall part 30, respectively. The thickness g thereby preferably likewise results in the radial direction. A protrusion 35 can thus be limited on the inside as well as radially on the outside by means of an axial extension of the receptacle wall 8, which has an outer surface 36 and an inner surface 37, or of the radially inner wall part 30, respectively.

In relation to a projection into the plate plane E, the protrusion 35 can run so as to be adapted radially inwards to the curvature of the inner surface 37. The protrusion 35 can accordingly merge into the inner surface 37 radially on the inside in the same plane. Directed radially outwards, the protrusion 35 can be designed in the shape of the segment of a circle in a perpendicular projection onto the plate plane E (see FIG. 20).

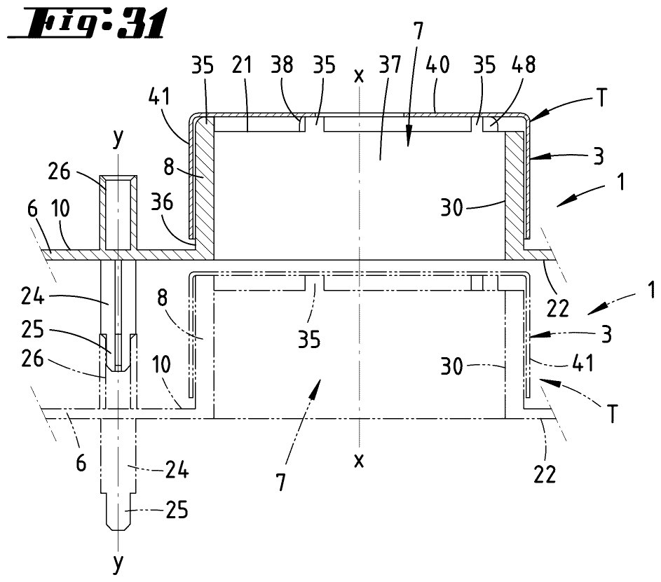

The protrusion 35 can also slope towards the outer surface 36 of the receptacle wall 8 or of the radially inner wall part 30, respectively. This can be attained, for example, by forming a rounding 38 according to the illustration in FIG. 25. Viewed in the circumferential direction U, the transition of such a rounding 38 into the lateral boundary surfaces 48 of the protrusion 35 can be flanked by edge recesses 39, for releasing a rounded web 49 of the protrusion 35 instead of the rounding 38 (see FIG. 26).

The protrusion 35 can have a free axial height h above the first front surface 21 which can correspond to approximately 1 to approximate 2 times the thickness g of the receptacle wall 8 or of the radially inner wall part 30, respectively.

The circumferential extension k of the protrusion 35 can be adapted to the thickness g of the receptacle wall 8 or of the radially inner wall part 30, respectively.

A minimization of the contact surface of the part T to be received is also attained in the case of this design. For example, a receptacle 7 designed according to FIGS. 17 to 32 can serve the purpose of holding caps 3 in a transport-safe manner, which can support themselves point by point with the inner side of the cap top 40 on the protrusions 35. By means of the protrusions 35 provided so as to be distributed over the circumference, free span top regions of the cap 3 to be held result in the circumferential direction U between two protrusions 35, which are exposed in particular for carrying out a sterilization process.

According to the illustration in FIG. 19A, the cap wall 41 can dip into the slit 32 between the wall parts 30 and 31 of the double wall or can engage over the receptacle wall in the case of a design of the receptacle 7 according to the fourth embodiment (see FIG. 31).

For example in the case of a design of the receptacle 7 with a double wall directed downwards according to FIGS. 17 to 22B, pin-like support feet 24 can be molded on the second plate side 22, the axial length of which is selected so that a direct support on the vessel bottom 28 or on a plate part 6 of a further holding part 1 can be attained (see FIGS. 18, 19A, and 19B).

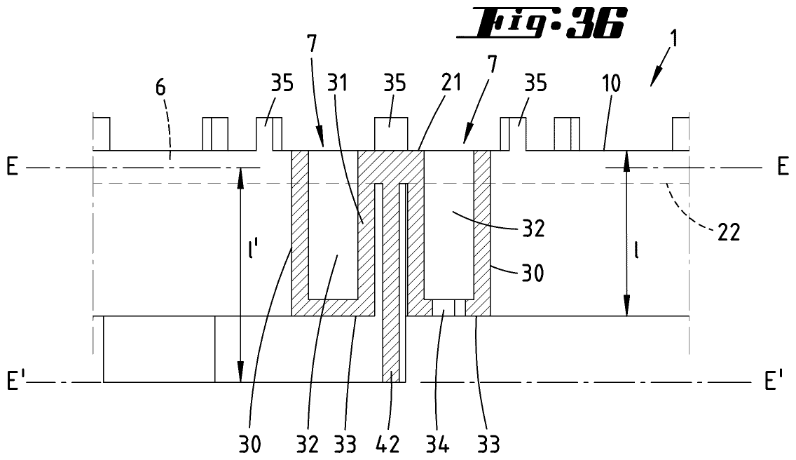

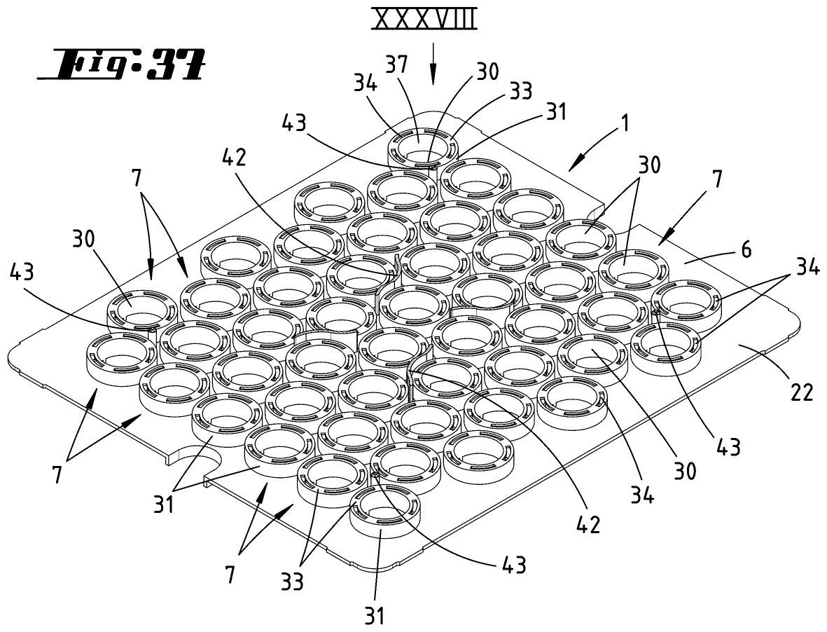

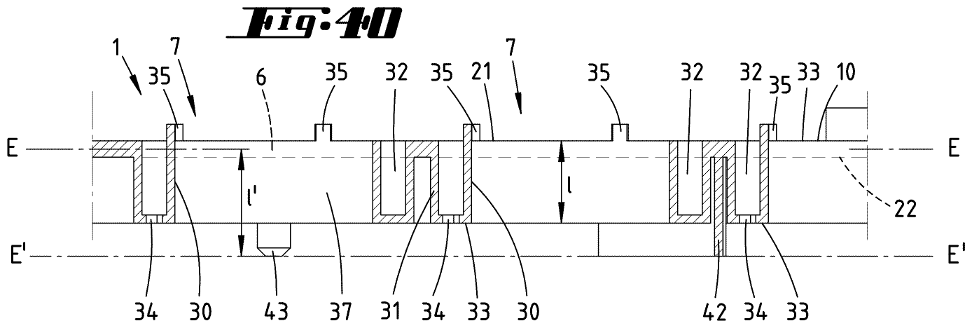

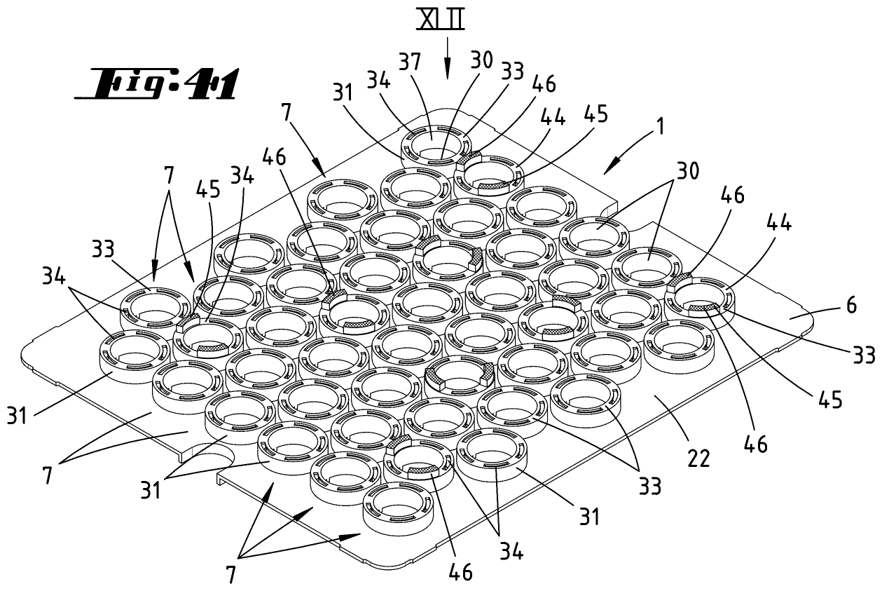

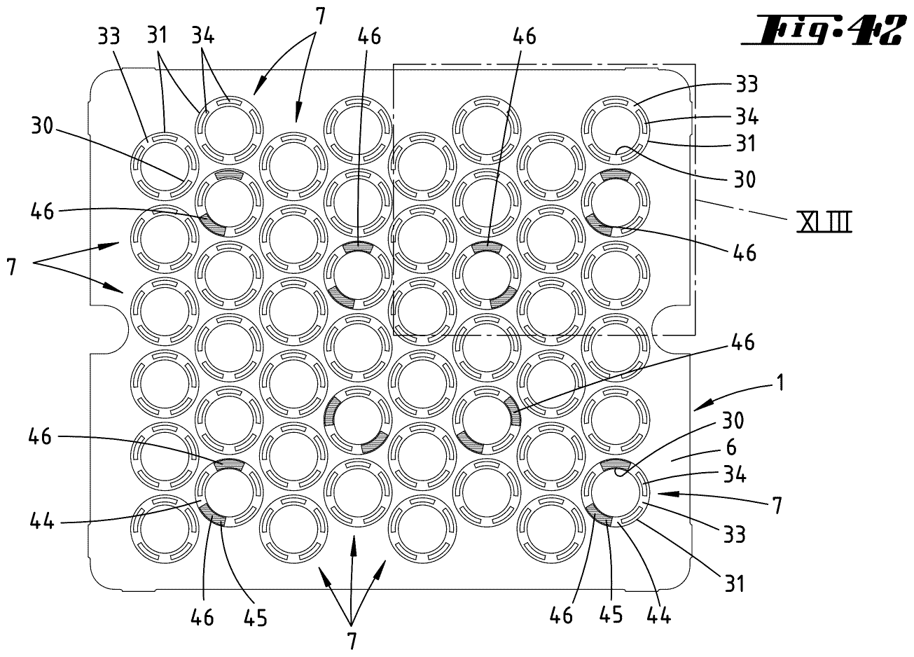

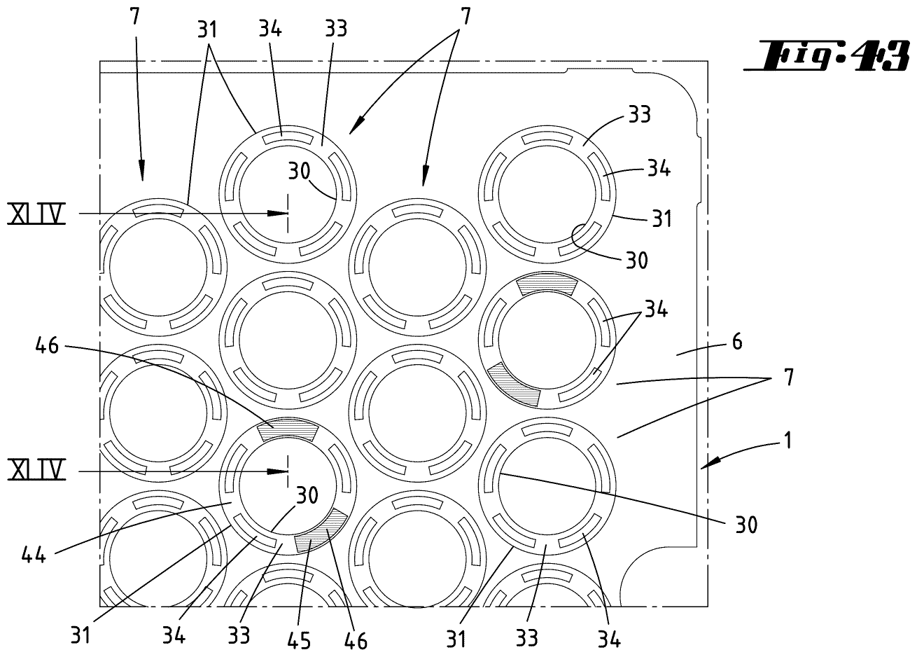

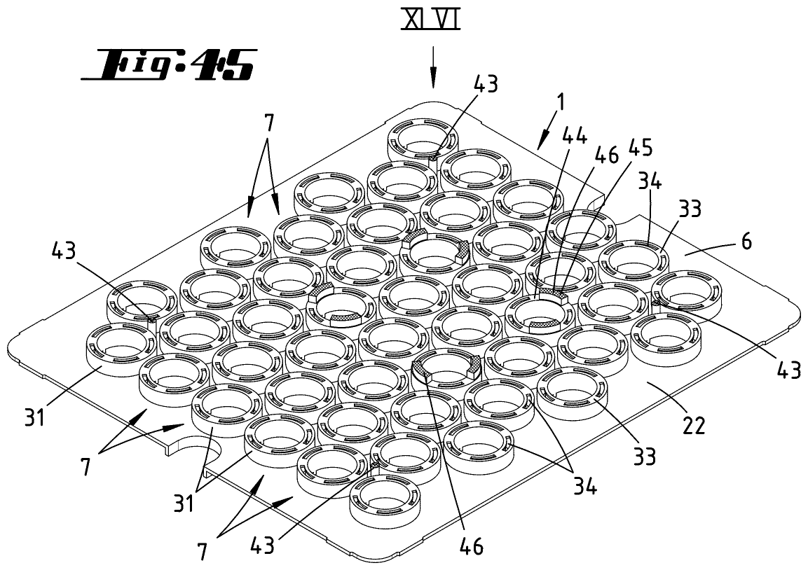

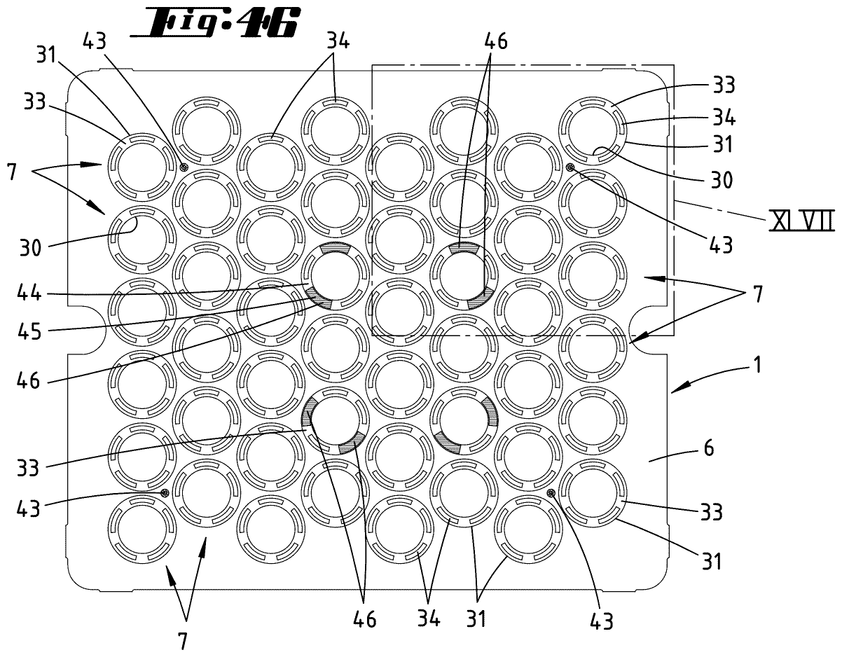

The FIGS. 33 to 48 show further embodiments with receptacles 7, which have a double wall according to the embodiment in FIGS. 17 to 22B.

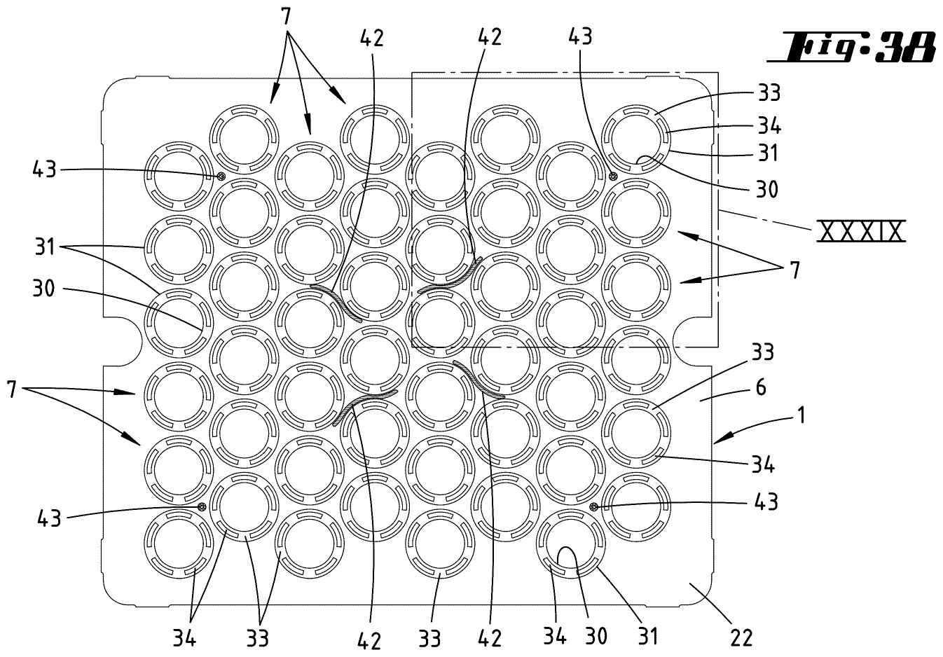

Viewed in the direction of the central axis x, the receptacles 7 have a first height l starting at the first front surface 21 all the way to the first plate side 10. A support wall 42 can extend between the receptacles 7, which are arranged spaced apart from one another. This support wall 42 preferably has a second height l′, which is larger than the first height l (see FIG. 36). The second height l′ can thus correspond, for example, to 1.2 to 3 times, further for example approximately to 1.5 times the first height l.

The support wall 42 can have a longitudinal extension p, which can correspond approximately to three times to four times a free diameter d of a receptacle 7 in the FIGS. 33 to 36. The longitudinal extension p is thereby determined in a perpendicular projection onto the plate plane E and along a straight line running parallel to the plate plane E connecting the beginning and the end of the support wall 42 (see FIG. 35).

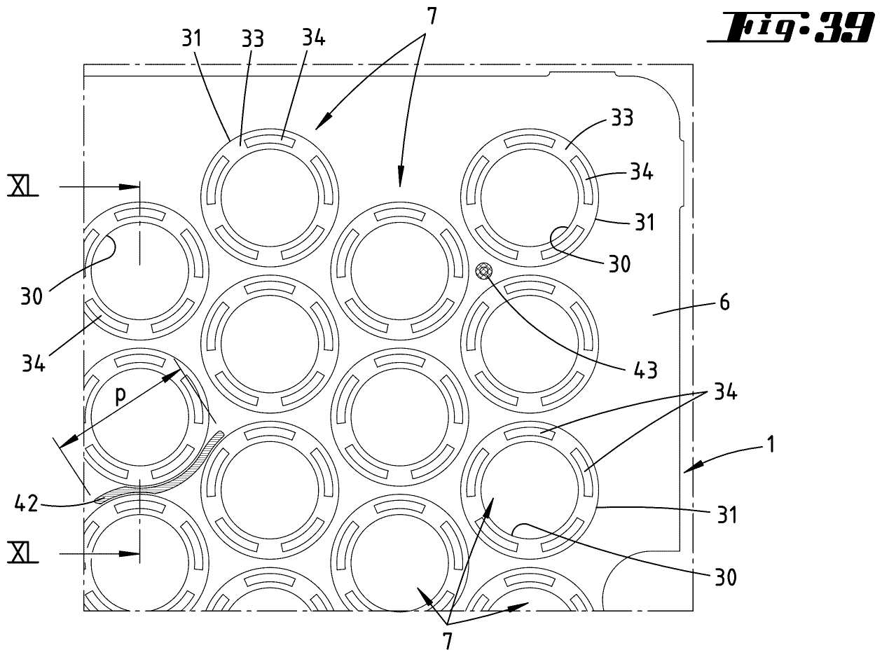

The embodiment of FIGS. 37 to 40 likewise shows a support wall 42, which extends between the receptacles 7, but which has a longitudinal extension p, which corresponds only approximately to 1 to 1.5 times the free diameter d of a receptacle 7.

As can be seen, the support wall 42 can extend in a wave-like manner in its longitudinal direction between the receptacles 7.

Several support walls 42, which are not connected to one another, can be formed on the holding part 1 in an advantageous manner. These support walls 42 can be provided so as to be distributed freely over the second plate side 22. Such support walls 42 can also be formed on the first plate side 10.

In the embodiment of FIGS. 33 to 36, the support walls 42, which are provided with a larger longitudinal extension p, are provided in a central plate region of the holding part 1. Solely a support via these support walls 42 can be at hand.

If shorter support walls 42 are provided, as they are shown in FIGS. 37 to 40, further support posts 43 can be provided, assigned to an edge region of the plate part 6. Starting at the plate plane E, they extend over the same second height l′ as the support walls 42 in preferred parallel alignment to the central axis x. A support plane E′ accordingly results, which is defined jointly by the support walls 42 and the support posts 43.

Further alternatively to or in combination with the support walls 42 and/or the support posts 43, the front surface 33 can be formed in a stepped manner in the circumferential direction U. Front surface planes 44 and 45, which are spaced apart differently from the plate plane E, result accordingly. For example, two front surface planes 44 and 45 can thus be at hand, with a first front surface plane 44 closer to the plate plane E and a second front surface plane 45 further away from the plate plane E. A support protrusion 46, which protrudes beyond the first front surface plane 44, can be formed by means of this plane offset.

In a possible design, the second front surface plane 45 can assume a distance from the plate plane E, which corresponds to the second height l′, for example of the support walls 42 and/or of the support posts 43.

Several support protrusions 46 can be formed over the circumference. In the case of the exemplary embodiments illustrated in FIGS. 41 to 48, two support protrusions 46 are thus provided, which are spaced apart from one another in the circumferential direction U.

The support protrusions 46 of at least the same receptacle 7 can also be of identical design.

In the central region as well as assigned to an edge region of the holding part 1, the embodiment of FIGS. 41 to 44 has receptacles 7 with protrusions 46 of this type, so that a support solely on these support protrusions 46 can be attained.

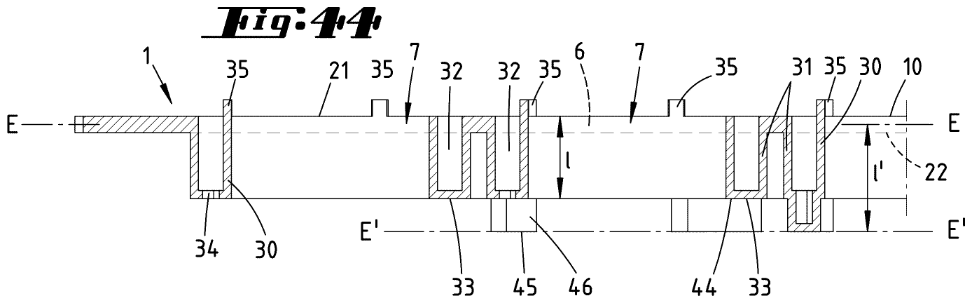

In the case of the embodiment according to FIGS. 45 to 48, in contrast, only centrally provided receptacles 7 are provided with such support protrusion 46, while support posts 43 can be arranged to support the edge regions.

As can further be seen from the illustrations, the support protrusions 46 can be formed so as to be closed in the region of their second front surface planes 45. The openings 34 preferably result only in the first front surface plane 44.

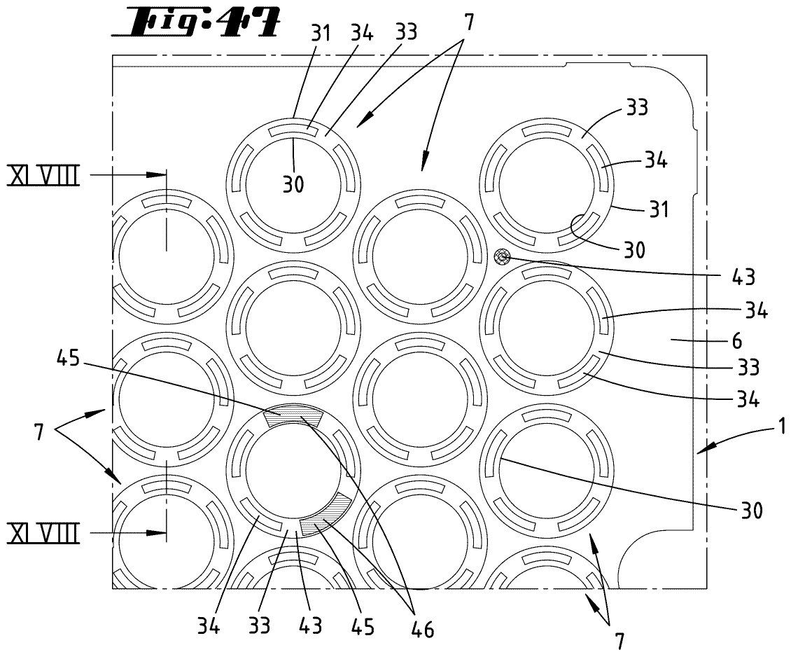

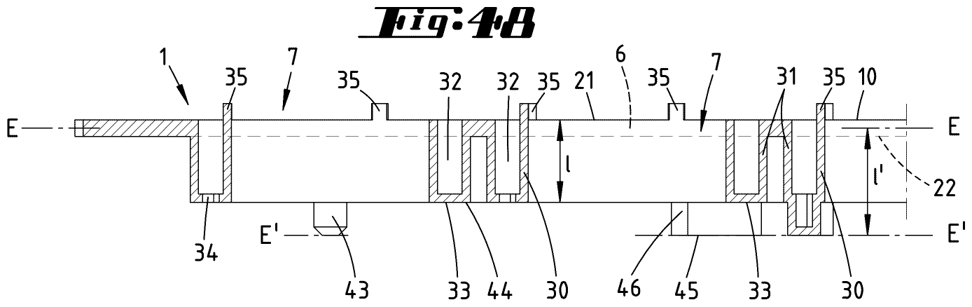

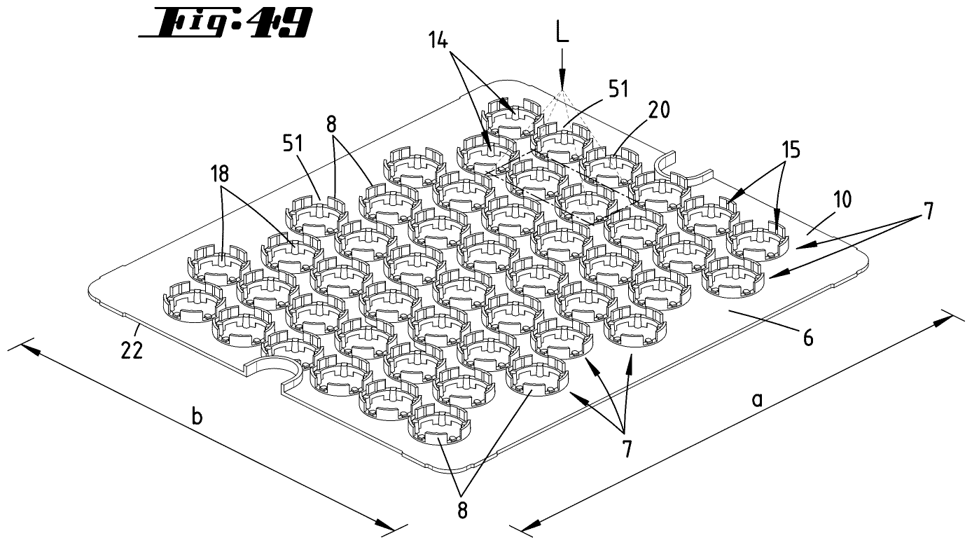

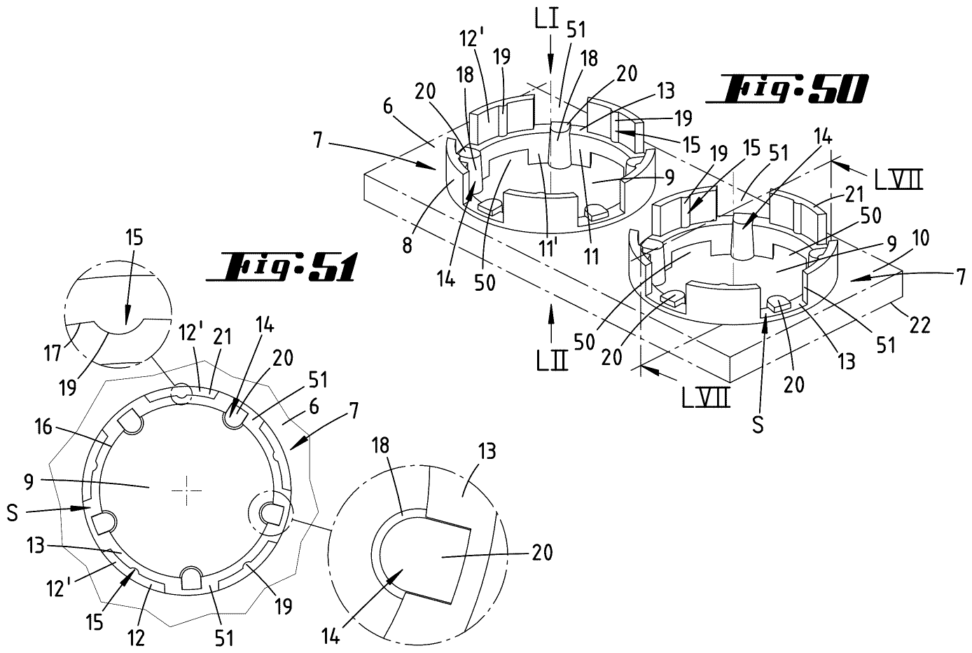

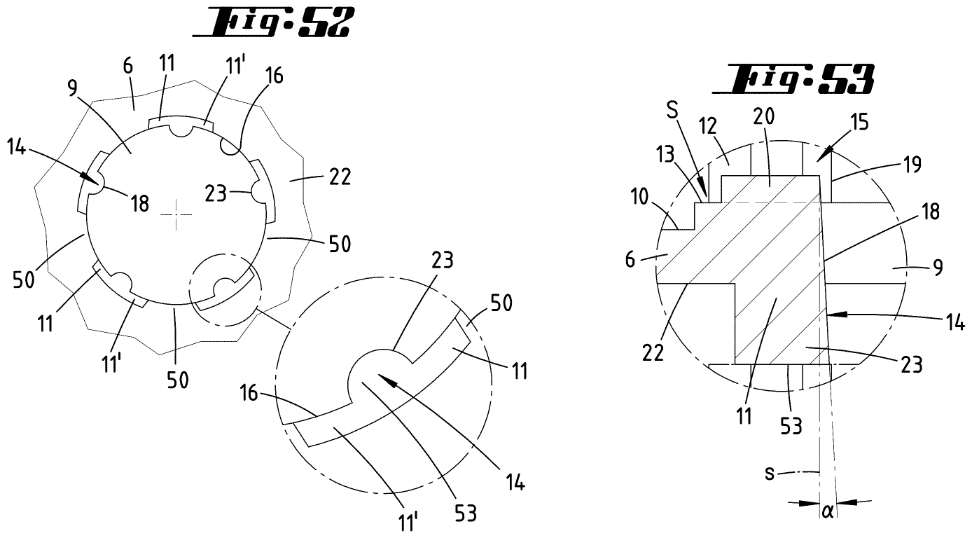

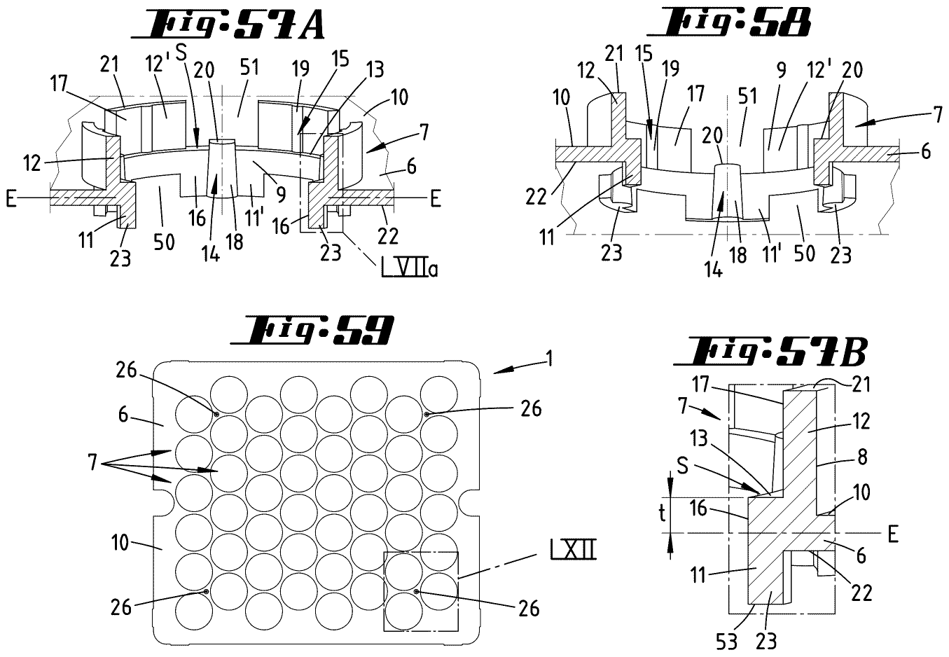

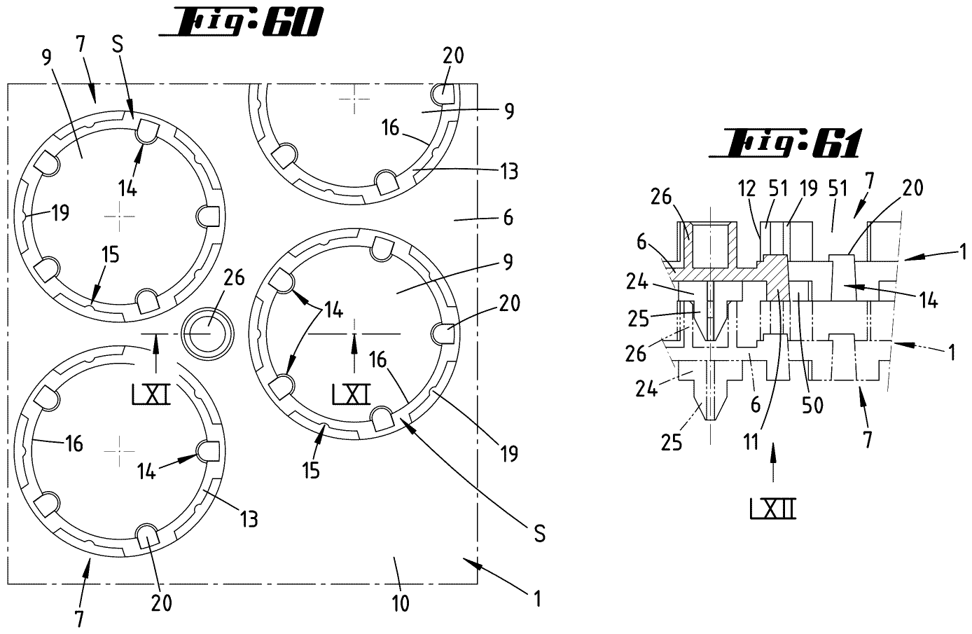

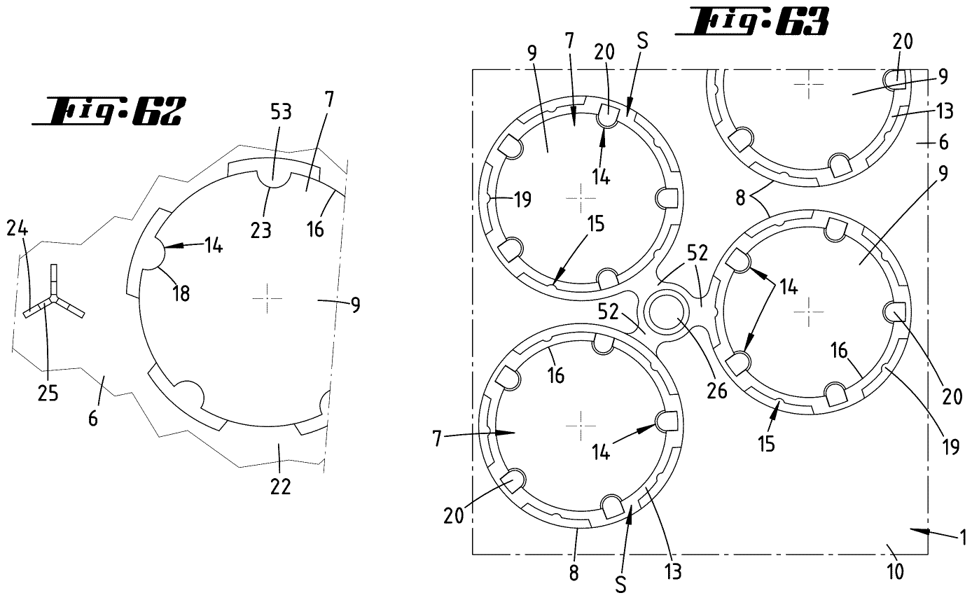

FIGS. 49 to 63 show further embodiments in which the receptacle wall 8 extends beyond both the first plate side 10 and the second plate side 22 (see sectional view in FIG. 56).

These further embodiments are essentially based on the embodiment shown in FIGS. 1 to 15. However, a further supporting area with the second holding formations 15 is formed essentially above the first plate side 10. A supporting area with the first holding formations 14 is formed essentially in the plane of the plate part 6 and below the second plate side 22.

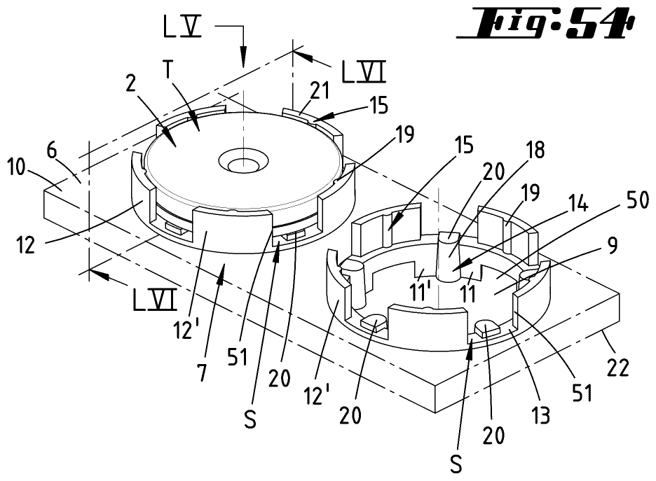

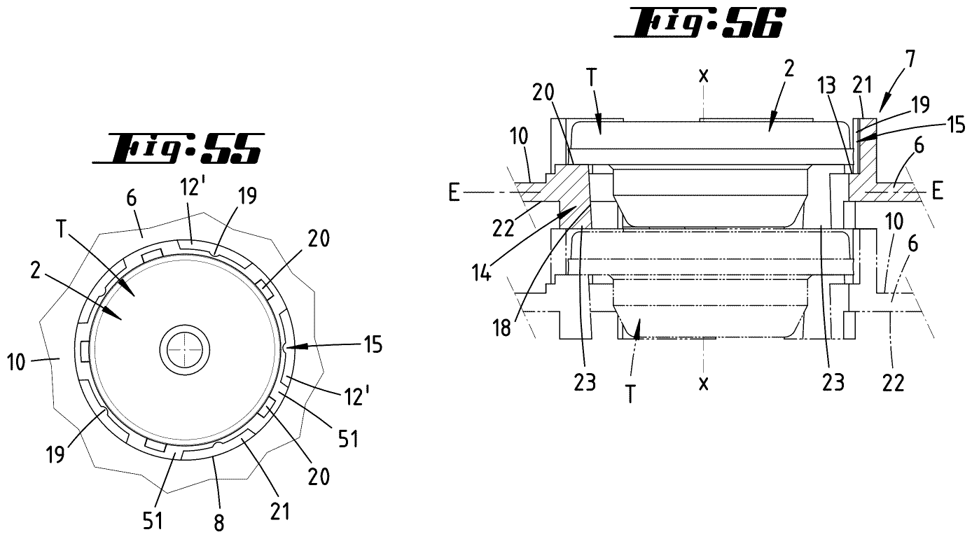

It can be seen from the enlargement in FIG. 57B that in this embodiment, as in the first embodiment described with reference to FIGS. 1 to 15, the first wall section 11 also extends in the axial direction from the underside of the first supporting foot 23 and/or from the second plate side 22, passing through the plate plane E, to the step S or the step surface 13 formed by the step S. The second wall section 12 extends in the axial direction from the step surface 13 to the first front surface 21, and accordingly also begins here at a distance t from the plate plane E. As to possible values of the distance t, reference is made to what is outlined above in view of FIG. 5B.

The receptacle wall 8 is preferably interrupted in the circumferential direction U both on the upper side of the first plate side 10 and on the underside of the second plate side 22.

There are first free spaces 50 in the area of the first wall section 11 and second free spaces 51 in the area of the second wall section 12. These free spaces can be evenly distributed around the circumference. The free spaces 50 or 51 can also be of essentially the same shape in relation to a wall section 11 or 12.

The first free spaces 50 extend into the direction to the plate plane preferably to the second plate side 22 of the plate part 6. They can also extend starting from the plate side 22 to a level corresponding to the front surface 53 of the first support feet 23. In-between neighboring parts of first wall sections, there is no limitation of the free space 50 in the direction of the extension of the first wall sections seen from the plate plane. This is usually a vertical direction (seen downwards).

The second free spaces 51 are limited in the axial direction by the step surface 13, seen in direction to the plate plane and possibly by an elevation 20 projecting into the step surface 13. Starting from this boundary, a second free space 51 can extend in the direction of the second wall sections up to a level corresponding to the first front surface 21 of the receptacle wall 8. Further in a direction vertical to the plate plane, which means vertically upwards, the second free space 51 is not limited.

The free spaces 50 and 51 extend—with respect to a projection of the free spaces 50 and 51 into the plate plane E—offset from one another in the circumferential direction U. In this projection, a second free space 51 can essentially overlap with a section 11′ of the receptacle wall 8 or of the first wall section 11 between two first free spaces 50 that bound these sections 11′ in the circumferential direction U. With regard to the second wall section 12, there are sections 12′ that are bounded in the circumferential direction U by second free spaces 51.

The radial free spaces 50 and/or 51 allow for improved gripping of the parts T located in the receptacles 7.

The sections 11′ and 12′ carry the first and second holding formations 14 and 15, respectively, essentially in accordance with the embodiment described on the basis of FIGS. 1 to 15.

The first support feet 23 can be formed directly through the lower surface of the first wall section 11, in particular the section 11′, and correspondingly preferably not exposed, but rather at the same level as this lower surface, as shown in this embodiment.

The second support feet 24 or the insertion sections 25 and/or the insertion receptacles 26 can be arranged in a gusset area between the receptacles 7 in a free-standing manner, as shown for example in FIGS. 16, 29 and 60.

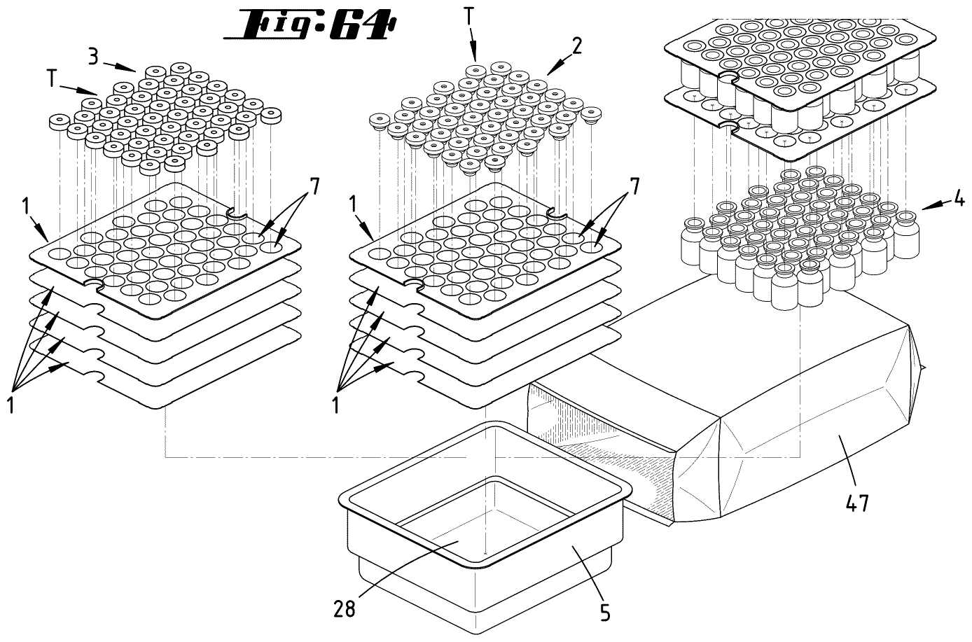

Alternatively, the insertion sections 25 and/or the insertion receptacles 26 can be connected to the receptacle walls 8 of the nearest receptacles 7 in a radially outward direction, as shown in FIG. 63. Correspondingly between an insertion section 25 and/or an insertion receptacle 26 and the receptacle wall 8 of the receptacle 7, connecting bridges 52 can arise that extend essentially radially.

Also, tubular second support feet 24 in the manner of the further described support posts 43 (see for example FIG. 45) can be placed between the receptacles 7 only for the purpose of spacing the holding parts 1 when stacked as loose parts.

Although only a few embodiments of the present invention have been shown and described, it is to be understood that many changes and modifications may be made thereunto without departing from the spirit and scope of the invention.

### LIST OF REFERENCE NUMERALS

1 holding part
2 stopper
3 cap
4 vessel
5 transport vessel
6 plate part
7 receptacle
8 receptacle wall
9 receptacle opening
10 first plate side
11 first wall section
11 section
12 second wall section
12′ section
13 step surface
14 first holding formation
15 second holding formation
16 wall inner surface
17 wall inner surface
18 holding surface
19 holding surface
20 elevation
21 first front surface
22 second plate side
23 first support foot
24 second support foot
25 insertion section
26 insertion receptacle
27 step section
28 bottom
29 connecting bridge
30 inner wall part
31 outer wall part
32 slit
33 front surface
34 opening
35 protrusion
36 outer surface
37 inner surface
38 rounding
39 edge recess
40 cap top
41 cap wall
42 support wall
43 support post
44 first front surface plane
45 second front surface plane
46 support protrusion
47 bag
48 boundary surface
49 web
50 first free space
51 second free space
52 bridge
53 second front surface
a length
b width
c protrusion dimension
c′ protrusion dimension
d diameter
d′ diameter
e dimension
f radial dimension
g thickness
h height
k circumferential extension
l first height
l′ second height
p longitudinal extension
5 perpendicular
t distance
x central axis
y central axis
E plate plane
E′ support plane
S step
T part
U circumferential direction
α cone angle
β angle
δ angle

1. A plate-like holding part (1) for holding parts (T), such as stoppers (2) or caps (3) for pharmaceutical vessels (4), comprising a plate part (6) having a plate plane (E), wherein a receptacle (7) extending perpendicular to the plate plane (E) is formed on a first plate side (10) of the plate part (6), with a circular ring-shaped receptacle wall (8), wherein the receptacle wall (8) is formed with a step(S), with a first wall section (11) penetrated by the plate plane (E) and a second wall section (12) adjoining the step(S), which begins at a distance (t) from the plate plane (E).

2. The holding part according to claim 1, wherein the step(S) has a step surface (13) extending essentially parallel to the plate plane (E).

3. The holding part according claim 1, wherein the first wall section (11) has several first holding formations (14), which protrude radially inwards.

4. The holding part according to claim 1, wherein the second wall section (12) has several second holding formations (15), which protrude radially inwards.

5-6. (canceled)

7. The holding part according to claim 3, wherein first holding formations (14) widen conically starting at a step surface (13).

8. The holding part according to claim 2, wherein the step surface (13) has several pedestal-like elevations (20) over a circumference.

9.-10. (canceled)

11. The holding part according to claim 1, wherein on a second plate side (22) lying opposite to the receptacle wall (8), several support feet (23) are formed on the plate part (6).

12.-13. (canceled)

14. The holding part according to claim 1, wherein the first wall section (11) is interrupted over the circumference by first free spaces (50).

15. The holding part according to claim 14, wherein the first free spaces (50) are limited by a second plate side (22) of the plate part (6).

16. The holding part according to claim 1, wherein the second wall section (12) is interrupted over the circumference by second free spaces (51), wherein the step(S) has a step surface (13) extending essentially parallel to the plate plane (E) and wherein the second free spaces (51) are limited by the step surface (13) and optionally by pedestal-like elevations (20) projecting into the step surface (13).

17. (canceled)

18. A plate-like holding part (1) for holding parts (T), such as stoppers (2) or caps (3) for pharmaceutical vessels (4), comprising a plate part (6) having a plate plane (E), wherein a receptacle (7) extending perpendicular to the plate plane (E) is formed on a first plate side (10) of the plate part (6), with a circular ring-shaped receptacle wall (8), wherein a front surface (21) of the receptacle wall, which runs in an approximately plate-parallel manner, has several protrusions (35) distributed over the circumference.

19. The holding part according to claim 18, wherein each protrusion (35) is adapted in a radial direction to a thickness (g) of the receptacle wall (8).

20-41. (canceled)

42. The holding part according to claim 18, wherein a circumferential extension (k) of the protrusion (35) corresponds approximately to a thickness (g) of the receptacle wall (8).

43. The holding part according to claim 18, wherein on a second plate side (22) lying opposite the receptacle wall (8) or on the first plate side (10), the plate part (6) has one or several support feet (24).

44. The holding part according to claim 18, wherein a plurality of receptacles (7) extending perpendicular to the plate plane (E) are formed on the plate part (6), each with a circular ring-shaped receptacle wall (8), wherein the receptacle walls (8) have an essentially identical first height (l) and wherein the receptacle wall (8) is formed as a double wall, with two concentrically circular wall parts (30, 31).

45. A plate-like holding part (1) for holding parts (T), such as stoppers (2) or caps (3) for pharmaceutical vessels (4), comprising a plate part (6) having a plate plane (E), wherein a plurality of receptacles (7) extending perpendicular to the plate plane (E) is formed on the plate part (6), each with a circular ring-shaped receptacle wall (8), wherein the receptacle walls (8) have an essentially identical first height (l), wherein a support wall (42) extends between the receptacles (7), which has a second height (l′), wherein the second height (l′) is larger than the first height (l), and wherein the support wall (42) has a longitudinal extension (p), which corresponds to half of a free diameter (d) or more of one of the receptacles (7).

46. The holding part according to claim 45, wherein the longitudinal extension (p) corresponds up to three free diameters (d) or more of one of the receptacles (7).

47. The holding part according to claim 45, wherein several support walls (42), which are not connected to one another, are formed.

48. The holding part according to claim 45, wherein at least one of the receptacle walls (8) is formed as double wall, with two concentrically circular wall parts (30, 31).

49. The holding part according to claim 48, wherein the wall parts (30, 31) are connected on their ends, which are spaced apart from the plate plane (E), by means of a front surface (33).

50. The holding part according to claim 48, wherein one of the wall parts (30, 31) merges into a protrusion (35) extending further in a direction vertical to the first or second plate side (10, 22) of the plate part (6).
